(19) United States
(12) Patent Application Publication
(10) Pub. No.: US 2025/0387523 A1
(43) Pub. Date: Dec. 25, 2025

(54) COMBINATION THERAPY OF RADIONUCLIDE COMPLEX

(71) Applicants: Novartis AG, Basel (CH); Advanced Accelerator Applications, Rueil-Malmaison (FR)

(72) Inventors: Paola Daniela AIMONE, Rixheim (FR); Daniela CHICCO, Bollengo (IT); Maurizio F. MARIANI, Ivrea (IT); Evren ZOR, Basel (CH)

(21) Appl. No.: 18/704,804

(22) PCT Filed: Oct. 27, 2022
(86) PCT No.: PCT/IB2022/060331

§ 371 Date: Apr. 25, 2024

Related U.S. Application Data
(60) Provisional application No. 63/272,447, filed on Oct. 27, 2021.

(51) Int. Cl. A61K 51/08; A61K 31/495

(52) U.S. Cl. CPC A61K 51/083; A61K 31/495; A61K 51/088; A61K 2121/00

(57) ABSTRACT

The present disclosure is directed to a method of treating glioblastoma in a subject in need thereof comprising administering to said subject an efficient amount of a radiopharmaceutical compound. The present disclosure is also directed to methods of treating glioblastoma in a subject in need thereof comprising administering to said subject an efficient amount of a radiopharmaceutical compound in combination with a step of irradiating the subject with an efficient dose of ionizing radiations, and optionally, with a therapeutically efficient amount of an alkylating agent, preferably temozolomide.

### FIELD OF THE INVENTION

The present invention relates to methods for treating glioblastoma in a subject in need thereof wherein a therapeutically efficient amount of said radiopharmaceutical compound is administered to said subject in combination with radiotherapy.

### BACKGROUND

Glioblastoma (GB) is the most commonly occurring malignant central nervous system (CNS) tumor accounting for 14.6% of all tumors (Ostrom Q T, Cioffi G, Gittleman H, et al (2019) CBTRUS Statistical Report: Primary Brain and Other Central Nervous System Tumors Diagnosed in the United States in 2012-2016. Neuro Oncol; 12 (S5): 1-100). It is an aggressive primary brain tumor, with a high mortality rate despite extensive efforts to develop new treatments. Currently, there are no curative treatment options for glioblastoma and despite rigorous therapeutic research, the survival rate of patients diagnosed with glioblastoma remains low. Median overall survival (OS) is approximately 15 months, and 5-year survival is less than 10% (Wen P Y, Weller M, Lee E Q, et al (2020) Glioblastoma in adults: a Society for Neuro-Oncology (SNO) and European Society of Neuro-Oncology (EANO) consensus review on current management and future directions. Neuro Oncol; 22 (8): 1073-113). Glioblastoma is one of the lowest long-term survival rate of malignant brain tumors with a 5-year overall relative survival of only 6.8% (Ostrom Q T, Cioffi G, Gittleman H, et al (2019) CBTRUS Statistical Report: Primary Brain and Other Central Nervous System Tumors Diagnosed in the United States in 2012-2016. Neuro Oncol; 12 (S5): 1-100).

The overall age-adjusted incidence of glioblastoma in the United States is 3.22/100 000 persons, is higher in males and increases with advanced age at diagnosis (Wen P Y, Weller M, Lee E Q, et al (2020) Glioblastoma in adults: a Society for Neuro-Oncology (SNO) and European Society of Neuro-Oncology (EANO) consensus review on current management and future directions. Neuro Oncol; 22 (8): 1073-113). Standard therapy for newly diagnosed glioblastoma patients begins with a surgical procedure intended to perform a maximal safe tumor resection (Nabors L B, Portnow J, Ahluwalia M, et al (2020) Central Nervous System Cancers, Version 3.2020, NCCN Clinical Practice Guidelines in Oncology. J Natl Compr Canc Netw p. 1537-1570). Glioblastoma is a radiosensitive tumor and radiation therapy (RT) has been considered to be the most important treatment modality for glioblastoma following surgery since 1980's.

The current standard of care in newly diagnosed patients is the combination of temozolomide (TMZ) (an oral alkylating agent) with radiotherapy (RT) which was approved based on the results of a large, randomized, phase III trial comparing radiotherapy (60 gy over 6 weeks) to radiotherapy plus concomitant daily temozolomide 75 mg/m2/day, followed by temozolomide maintenance 150 to 200 mg/m2/day for 5 consecutive days out of every 28-day cycle, for up to 6 cycles (Stupp R, Mason W P, van den Bent M J, et al (2005) Radiotherapy plus concomitant and adjuvant temozolomide for glioblastoma. N Engl J Med; 352:987-96). The addition of temozolomide to radiation therapy prolonged the median overall survival from 12.1 to 14.6 months.

The methylation of the promoter of the O-6-methylguanine-DNA methyltransferase (MGMT) gene in glioblastoma is both a prognostic and a predictive marker for response to treatment with alkylating agents. in a study including 206 newly diagnosed glioblastoma patients the overall survival of patients with MGMT promoter methylation was highly significant vs. those whose tumors did not have a methylated MGMT promoter (P<0.001; hazard ratio for death, 0.45). This study also showed that in patients with a methylated MGMT promoter, a survival benefit was observed in those treated with temozolomide and radiotherapy with a median survival of 21.7 months, as compared to 15.3 months in those treated with radiotherapy only (P=0.007). By contrast, in patients whose tumors were not methylated at the MGMT promoter, the difference in overall survival was not significant, with a median survival of 12.7 months in patients treated with temozolomide plus radiotherapy and 11.8 months in those treated with radiotherapy only (P=0.06) (Hegi M E, Diserens A C, Gorlia T, et al (2005) MGMT gene silencing and benefit from temozolomide in glioblastoma. N Engl J Med; 352:997-1003). Other trials also have shown that the presence of MGMT promoter methylation results in approximately 50% longer median survival for glioblastoma patients treated with temozolomide and in patients that lack MGMT promoter methylation. In this context, the use of temozolomide has no clinical benefit and brings unwanted toxicity in this group of patients. As such, withholding temozolomide from glioblastoma that lack MGMT promoter methylation became acceptable, especially in the context of clinical trials conducted recently (Wen P Y, Weller M, Lee E Q, et al (2020) Glioblastoma in adults: a Society for Neuro-Oncology (SNO) and European Society of Neuro-Oncology (EANO) consensus review on current management and future directions. Neuro Oncol; 22 (8): 1073-113).

Inevitably, almost all patients will experience disease recurrence, the median progression free survival (PFS) is approximately 6-10 months (Wen P Y, Weller M, Lee E Q, et al (2020) Glioblastoma in adults: a Society for Neuro-Oncology (SNO) and European Society of Neuro-Oncology (EANO) consensus review on current management and future directions. Neuro Oncol; 22 (8): 1073-113). Available therapeutic options for relapsed disease have limited survival benefit and there is no established sequence of therapies for recurrent glioblastoma. Treatment of recurrent glioblastoma is challenging because of the limited efficacy of available options and lack of established treatments options. Treatment guidelines recommend clinical trials as the preferred option for eligible patients (Nabors L B, Portnow J, Ahluwalia M, et al (2020) Central Nervous System Cancers, Version 3.2020, NCCN Clinical Practice Guidelines in Oncology. J Natl Compr Canc Netw p. 1537-1570; Wen P Y, Weller M, Lee E Q, et al (2020) Glioblastoma in adults: a Society for Neuro-Oncology (SNO) and European Society of Neuro-Oncology (EANO) consensus review on current management and future directions. Neuro Oncol; 22 (8): 1073-113). Surgery may have a role for symptomatic and/or large lesions. However, only patients who undergo complete or near complete resections have any survival benefit (Nam J Y, de Groot J F (2017) Treatment of Glioblastoma. J Oncol Pract; 13 (10): 629-39). Other treatment options include systemic therapy such as temozolomide re-challenge, nitrosoureas, bevacizumab, re-irradiation, and Tumor Treating Fields, none of which have been shown to prolong survival in randomized trials in this setting, or palliative care for patients with poor performance status. Single-agent nitrosoureas (carmustine, lomustine, and fotemustine) have been evaluated in recurrent glioblastoma. In a recent study, 437 patients were randomized 2:1 between lomustine as single agent and lomustine in combination with bevacizumab. Patients in the lomustine arm showed a median PFS of 1.5 months and OS of 8.6 months. The addition of bevacizumab to lomustine showed an improved median PFS of 4.2 months in the combination arm vs 1.5 months in lomustine arm (P<0.001); however median OS difference did not confer a survival difference with 9.1 months in the combination arm vs 8.6 months in the lomustine arm (Wick W, Gorlia T, Bendszus M, et al (2017) Lomustine and Bevacizumab in Progressive Glioblastoma. N Engl J Med; 377:1954-1963).

Pilot studies have assessed the activity of radiolabeled DOTA peptides inpatients with glioblastoma. Heute et al. reported the use of 90Y-DOTATOC in three grade IV recurrent glioblastoma patients (Heute D, Kostron H, von Guggenberg E, et al (2010) Response of recurrent high-grade glioma to treatment with (90) Y-DOTATOC. J Nucl Med; 51:397-400). Nemati et al. reported the use of 177Lu-DOTATATE in High-Grade Glioma (HGG) (Nemati R, Shooli H, Rekabpour S J, et al (2021) Feasibility and Therapeutic Potential of Peptide Receptor Radionuclide Therapy for High-Grade Gliomas. Clin Nucl Med; 46 (5): 389-95).

There is still a need to provide improved clinical treatments of glioblastoma.

### SUMMARY

The present disclosure relates to a method for treating glioblastoma in a subject in need thereof by administering a therapeutically efficient amount of a radiopharmaceutical compound to said subject in combination with radiotherapy, and optionally, temozolomide.

The present disclosure is provided in various aspects as outlined in the following:
1. A radiopharmaceutical compound for use in treating glioblastoma in a subject in need thereof wherein a therapeutically efficient amount of said radiopharmaceutical compound is administered to said subject preferably in combination with radiotherapy.
2. The radiopharmaceutical compound for use of embodiment 1, wherein said radiopharmaceutical compound is a compound of formula:

M-C—S—P wherein:
M is a radionuclide;
C is a chelating agent capable of chelating said radionuclide;
S is an optional spacer covalently linked between C and P;
P is a somatostatin receptor binding peptide covalently linked to C, either directly or indirectly via S.
3. The radiopharmaceutical compound for use of embodiment 1 or 2, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
4. The radiopharmaceutical compound for use of embodiments 1-3, wherein C is selected from DOTA (tetraxetan), trizoxetan, DOTAGA, DTPA, NTA, EDTA, DO3A, TETA, NOTA, NOTAGA, NODAGA, NODAPA, and AAZTA (e.g. AAZTA5) chelating agent, preferably is DOTA, DOTAGA, NOTA or DTPA chelating agent, and more preferably is DOTA chelating agent.
5. The radiopharmaceutical compound for use of embodiments 1-4, wherein P is selected from octreotide, octreotate, satoreotide, lanreotide, vapreotide, and pasireotide, preferably selected from octreotide and octreotate.
6. The radiopharmaceutical compound for use of embodiments 1-5, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
7. The radiopharmaceutical compound for use of embodiments 1-6, wherein the radiopharmaceutical compound is [¹⁷⁷Lu]Lu-DOTA-TOC (¹⁷⁷Lu-edotreotide) or [¹⁷⁷Lu]Lu-DOTA-TATE (¹⁷⁷Lu-oxodotreotide), more preferably [¹⁷⁷Lu]Lu-DOTA-TATE (¹⁷⁷Lu-oxodotreotide).
8. The radiopharmaceutical compound for use of embodiments 1-7, wherein said radiopharmaceutical compound is administered to said subject in combination with radiotherapy and with a therapeutically efficient amount of an alkylating agent, preferably temozolomide.
9. The radiopharmaceutical compound for use of embodiment 8, wherein said alkylating agent, preferably temozolomide, is administered (during an induction phase) at a dose of between 50 to 100 mg/m²/day, preferably around 75 mg/m²/day each day for an initial period of from 4 to 8 weeks, preferably of from 5 to 7 weeks, more preferably of 6 weeks.
10. The radiopharmaceutical compound for use of embodiment 8 or 9, wherein both radiotherapy and the administration of the alkylating agent, preferably temozolomide, are initiated the same day.
11. The radiopharmaceutical compound for use of embodiments 8-10, wherein said alkylating agent, preferably temozolomide, is concomittantly administered with the radiotherapy without interruption (from the first day until the last day of radiotherapy).
12. The radiopharmaceutical compound for use of embodiments 8-11, wherein said alkylating agent, preferably temozolomide, is daily administered at a first daily dose (preferably 50-100 mg/m²/day, more preferably 75 mg/m²/day) during the concomitant administration with the radiotherapy, for example for a period of 6 weeks (+1 week), and at a second dose during a maintenance phase, following the concomitant administration with the radiotherapy for example, for a period up to 24 weeks, wherein said second daily dose is at least twice the first daily dose and wherein preferably said second dose is administered on each of day 1 to day 5 of a 28-days-cycle.
13. The radiopharmaceutical compound for use of embodiments 8-12, wherein said alkylating agent, preferably temozolomide, is administered during the maintenance phase at a dose of between 50 to 400 mg/m²/day, preferably between 75 to 300 mg/m²/day, more preferably between 150 to 200 mg/m²/day at each of day 1 to day 5 of a 28-days-cycle for 4-8 cycles, preferably for 5-7 cycles, more preferably for 6 cycles.
14. The radiopharmaceutical compound for use of embodiments 8-13, wherein said subject has been selected from subject with positive methylated O-6-methylguanine-DNA methyltransferase promoter status.
15. The radiopharmaceutical compound for use of embodiments 1-14, wherein said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).
16. The radiopharmaceutical compound for use of embodiments 1-15, wherein said radiopharmaceutical compound is administered 1 to 8 times, preferably 2 to 7 times, more preferably 4 to 6 times, wherein there is a treatment interval between every two administrations of said radiopharmaceutical compound.
17. The radiopharmaceutical compound for use of embodiments 1-16, wherein the administration of said radiopharmaceutical compound comprises a treatment interval of 2 weeks, or 3 weeks, or 4 weeks, or 5 weeks or even 6 weeks, preferably 3 and/or 4 weeks, more preferably every 3 weeks.
18. The radiopharmaceutical compound for use of embodiments 1-17, wherein a first dose of said radiopharmaceutical compound is administered 1 to 20 days, preferably 3 to 15 days, more preferably 7 to 10 days prior to initiation of the radiotherapy.
19. The radiopharmaceutical compound for use of embodiments 1-18, wherein said radiotherapy induction is conducted at a dose between 1 Gy to 4 Gy/day, preferably around 2 Gy/day during a period between 3 to 7 days, preferably around 5 days per week during a period between 4 to 8 weeks, preferably 6 weeks.
20. The radiopharmaceutical compound for use of embodiments 1-19, wherein said radiotherapy is conducted for 5 consecutive days followed by 2 days of rest for 6 consecutive weeks.
21. The radiopharmaceutical compound for use of embodiments 1-20, wherein said radiotherapy is whole-brain radiotherapy.
22. The radiopharmaceutical compound for use of embodiments 1-21, wherein said subject has been selected for the treatment by SPECT/CT or PET/CT or SPECT/MRI, PET/MRI imaging with the same radiopharmaceutical compound as defined for the treatment but wherein M is a radiometal suitable for imaging, preferably ⁶⁸Ga, ⁶⁷Ga or ⁶⁴Cu, more preferably ⁶⁸Ga.
23. The radiopharmaceutical compound for use of embodiments 1-22, wherein said subject is newly diagnosed with glioblastoma or suffers from recurrent glioblastoma.
24. The radiopharmaceutical compound for use of embodiments 1-23 wherein said subject is newly diagnosed with glioblastoma and has a positive methylated O-6-methylguanine-DNA methyltransferase promoter status, wherein said radiopharmaceutical compound is administered to said subject in combination with radiotherapy and an alkylating agent, preferably temozolomide, wherein a first dose of said radiopharmaceutical compound is administered preferably 7 to 10 days prior to initiation of the radiotherapy.
25. The radiopharmaceutical compound for use of embodiments 1-24 wherein said subject is newly diagnosed with glioblastoma and has a negative methylated O-6-methylguanine-DNA methyltransferase promoter status, wherein said radiopharmaceutical compound is administered to said subject in combination with radiotherapy but not in combination with other chemotherapeutic agents, such as temozolomide; and wherein the treatment interval between two administrations of said radiopharmaceutical compound is for the first two intervals 4 weeks, and for the third and any following intervals, 3 weeks; and wherein a first dose of said radiopharmaceutical compound is administered preferably 7 to 10 days prior to initiation of the radiotherapy.
26. A method of treating glioblastoma in a subject in need thereof comprising administering to said subject an efficient amount of a radiopharmaceutical compound preferably in combination with a step of irradiating the subject with an efficient dose of ionizing radiations.
27. The method of embodiment 26, wherein said radiopharmaceutical compound is a compound of formula:

M-C—S—P wherein:
M is a radionuclide;
C is a chelating agent capable of chelating said radionuclide;
S is an optional spacer covalently linked between C and P;
P is a somatostatin receptor binding peptide covalently linked to C, either directly or indirectly via S.
28. The method of embodiment 26 or 27, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
29. The method of embodiments 26-28, wherein C is selected from DOTA (tetraxetan), trizoxetan, DOTAGA, DTPA, NTA, EDTA, DO3A, TETA, NOTA, NOTAGA, NODAGA, NODASA, NODAPA, and AAZTA (e.g. AAZTA5) chelating agent, preferably is DOTA, DOTAGA, NOTA or DTPA chelating agent, and more preferably is DOTA chelating agent.
30. The method of embodiments 26-29, wherein P is selected from octreotide, octreotate, satoreotide, lanreotide, vapreotide, and pasireotide, preferably selected from octreotide and octreotate.
31. The method of embodiments 26-30, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
32. The method of embodiments 26-31, wherein the radiopharmaceutical compound is [¹⁷⁷Lu]Lu-DOTA-TOC (¹⁷⁷Lu-edotreotide) or [¹⁷⁷Lu]Lu-DOTA-TATE (¹⁷⁷Lu-oxodotreotide), more preferably [¹⁷⁷Lu]Lu-DOTA-TATE (¹⁷⁷Lu-oxodotreotide).
33. The method of embodiments 26-32, said method further comprises administering a therapeutically efficient amount of an alkylating agent, preferably temozolomide.
34. The method of embodiment 33, wherein said alkylating agent, preferably temozolomide, is administered (during an induction phase) at a dose of between 50 to 100 mg/m²/day, preferably around 75 mg/m²/day each day for an initial period of from 4 to 8 weeks, preferably of from 5 to 7 weeks, more preferably of 6 weeks.
35. The method of embodiment 33 or 34, wherein both irradiation and the administration of the alkylating agent, preferably temozolomide, are initiated the same day.
36. The method of embodiments 33-35, wherein said alkylating agent, preferably temozolomide, is concomittantly administered with the irradiation without interruption (e.g. from the first day until the last day of irradiation).
37. The method of embodiments 33-36, wherein said alkylating agent, preferably temozolomide, is daily administered at a first daily dose (preferably 50-100 mg/m²/day, more preferably 75 mg/m²/day) during the concomitant administration with the radiotherapy, for example for a period of 6 weeks (+1 week), and at a second daily dose during a maintenance phase, following the concomitant administration with the radiotherapy for example, for a period up to 24 weeks, wherein said second daily dose is at least twice the first daily dose and wherein preferably said second dose is administered on each of day 1 to day 5 of a 28-days-cycle.
38. The method of embodiments 33-37, wherein said alkylating agent, preferably temozolomide, is administered during the maintenance phase at a dose of between 50 to 400 mg/m²/day, preferably between 75 to 300 mg/m²/day, more preferably between 150 to 200 mg/m²/day at each of day 1 to day 5 of a 28-days-cycle for 4-8 cycles, preferably for 5-7 cycles, more preferably for 6 cycles.
39. The method of embodiments 33-38, wherein said subject has been selected from subject with positive methylated O-6-methylguanine-DNA methyltransferase promoter status.
40. The method of embodiments 26-39, wherein said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).
41. The method of embodiments 26-40, wherein said radiopharmaceutical compound is administered 1 to 8 times, preferably 2 to 7 times, more preferably 4 to 6 times, wherein there is a treatment interval between every two administrations of said radiopharmaceutical compound.
42. The method of embodiments 26-41, wherein said radiopharmaceutical compound comprises a treatment interval of 2 weeks, or 3 weeks, or 4 weeks, or 5 weeks or even 6 weeks, preferably 3 and/or 4 weeks, more preferably every 3 weeks.
43. The method of embodiments 26-42, wherein a first dose of said radiopharmaceutical compound is administered 1 to 20 days, preferably 3 to 15 days, more preferably 7 to 10 days prior to initiation of the irradiation.
44. The method of embodiments 26-33, wherein said irradiation induction is conducted at a dose between 1 Gy to 4 Gy/day, preferably around 2 Gy/day during a period between 3 to 7 days, preferably around 5 days per week during a period between 4 to 8 weeks, preferably 6 weeks.
45. The method of embodiments 26-44, wherein said irradiation is conducted for 5 consecutive days followed by 2 days of rest for 6 consecutive weeks.
46. The method of embodiments 26-45, wherein said irradiation is whole-brain irradiation.
47. The method of embodiments 26-46, wherein said subject has been selected for the treatment by SPECT/CT or PET/CT or SPECT/MRI, PET/MRI imaging with the same radiopharmaceutical compound as defined for the treatment but with a radiometal suitable for imaging instead of ¹⁷⁷Lu, preferably ⁶⁸Ga, ⁶⁷Ga or 6⁴Cu, more preferably ⁶⁸Ga, by evaluating said radiopharmaceutical compound suitable for imaging uptake in said subject.
48. The method of embodiments 26-47, wherein said subject is newly diagnosed with glioblastoma or suffers from recurrent glioblastoma.
49. The method of embodiments 26-48, wherein said subject is newly diagnosed with glioblastoma and has a positive methylated O-6-methylguanine-DNA methyltransferase promoter status, wherein said radiopharmaceutical compound is administered to said subject in combination with radiotherapy and an alkylating agent, preferably temozolomide, wherein a first dose of said radiopharmaceutical compound is administered preferably 7 to 10 days prior to initiation of the irradiation.
50. The method of embodiments 26-49, wherein said subject is newly diagnosed with glioblastoma and has a negative methylated O-6-methylguanine-DNA methyltransferase promoter status, wherein said radiopharmaceutical compound is administered to said subject in combination with radiotherapy but not in combination with other chemotherapeutic agent, such as temozolomide; and wherein the treatment interval between two administrations of said radiopharmaceutical compound is for the first two intervals 4 weeks, and for the third and any following intervals, 3 weeks; wherein a first dose of said radiopharmaceutical compound is administered preferably 7 to 10 days prior to initiation of the irradiation.
51. Use of radiopharmaceutical compound in the preparation of a drug for use in treating glioblastoma in a subject in need thereof wherein a therapeutically efficient amount of said radiopharmaceutical compound is administered to said subject preferably in combination with radiotherapy.
52. Use of embodiment 51, wherein said radiopharmaceutical compound is a compound of formula:

M-C—S—P wherein:
M is a radionuclide;
C is a chelating agent capable of chelating said radionuclide;
S is an optional spacer covalently linked between C and P;
P is a somatostatin receptor binding peptide covalently linked to C, either directly or indirectly via S.
53. Use of embodiment 51 or 52, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
54. Use of embodiments 51-53, wherein C is selected from DOTA (tetraxetan), trizoxetan, DOTAGA, DTPA, NTA, EDTA, DO3A, TETA, NOTA, NOTAGA, NODAGA, NODASA, NODAPA, and AAZTA (e.g. AAZTA5) chelating agent, preferably is DOTA, DOTAGA, NOTA or DTPA chelating agent, and more preferably is DOTA chelating agent.
55. Use of embodiments 51-54, wherein P is selected from octreotide, octreotate, satoreotide, lanreotide, vapreotide, and pasireotide, preferably selected from octreotide and octreotate.
56. Use of embodiments 51-55, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE.
57. Use of embodiments 51-56, wherein the radiopharmaceutical compound is [¹⁷⁷Lu]Lu-DOTA-TOC (¹⁷⁷Lu-edotreotide) or [¹⁷⁷Lu]Lu-DOTA-TATE (¹⁷⁷Lu-oxodotreotide), more preferably [¹⁷⁷Lu]Lu-DOTA-TATE (¹⁷⁷Lu-oxodotreotide).
58. Use of embodiment 51-57, wherein said radiopharmaceutical compound is administered to said subject in combination with radiotherapy and with a therapeutically efficient amount of an alkylating agent, preferably temozolomide.
59. Use of embodiment 58, wherein said alkylating agent, preferably temozolomide, is administered (during an induction phase) at a dose of between 50 to 100 mg/m²/day, preferably around 75 mg/m²/day each day for an initial period of from 4 to 8 weeks, preferably of from 5 to 7 weeks, more preferably of 6 weeks.
60. Use of embodiments 58 or 59, wherein both radiotherapy and the administration of the alkylating agent, preferably temozolomide, are initiated the same day.
61. Use of embodiments 58-60, wherein said alkylating agent, preferably temozolomide, is concomittantly administered with the radiotherapy without interruption (e.g. from the first day until the last day of radiotherapy).
62. Use of embodiments 58-61, wherein said alkylating agent, preferably temozolomide, is daily administered at a first daily dose (preferably 50-100 mg/m²/day, more preferably 75 mg/m²/day) during the concomitant administration with the radiotherapy, for example for a period of 6 weeks (+1 week), and at a second daily dose during a maintenance phase, following the concomitant administration with the radiotherapy for example, for a period up to 24 weeks, wherein said second daily dose is at least twice the first daily dose and wherein preferably said second dose is administered on each of day 1 to day 5 of a 28-days-cycle.
63. Use of embodiments 58-62, wherein said alkylating agent, preferably temozolomide, is administered during the maintenance phase at a dose of between 50 to 400 mg/m²/day, preferably between 75 to 300 mg/m²/day, more preferably between 150 to 200 mg/m²/day at each of day 1 to day 5 of a 28-days-cycle for 4-8 cycles, preferably for 5-7 cycles, more preferably for 6 cycles.
64. Use of embodiments 58-63, wherein said subject has been selected from subject with positive methylated O-6-methylguanine-DNA methyltransferase promoter status.
65. Use of embodiments 51-64, wherein said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).
66. Use of embodiments 51-65, wherein said radiopharmaceutical compound is administered 1 to 8 times, preferably 2 to 7 times, more preferably 4 to 6 times, wherein there is a treatment interval between every two administrations of said radiopharmaceutical compound.
67. Use of embodiments 51-66, wherein the administration of said radiopharmaceutical compound comprises a treatment interval of 2 weeks, or 3 weeks, or 4 weeks, or 5 weeks or even 6 weeks, preferably 3 and/or 4 weeks, more preferably every 3 weeks.
68. Use of embodiments 51-67, wherein a first dose of said radiopharmaceutical compound is administered 1 to 20 days, preferably 3 to 15 days, more preferably 7 to 10 days prior to initiation of the radiotherapy.
69. Use of embodiments 51-68, wherein said radiotherapy induction is conducted at a dose between 1 Gy to 4 Gy/day, preferably around 2 Gy/day during a period between 3 to 7 days, preferably around 5 days per week during a period between 4 to 8 weeks, preferably 6 weeks.
70. Use of embodiments 51-69, wherein said radiotherapy is conducted for 5 consecutive days followed by 2 days of rest for 6 consecutive weeks.
71. Use of embodiments 51-70, wherein said radiotherapy is whole-brain radiotherapy.
72. Use of embodiments 51-71, wherein said subject has been selected for the treatment by SPECT/CT or PET/CT or SPECT/MRI, PET/MRI imaging with the same radiopharmaceutical compound as defined for the treatment but with a radiometal suitable for imaging with the same radiopharmaceutical compound as defined for the treatment but wherein M is a radiometal suitable for imaging, preferably ⁶⁸Ga, ⁶⁷Ga or ⁶⁴Cu, more preferably ⁶⁸Ga.
73. Use of embodiments 51-72, wherein said subject is newly diagnosed with glioblastoma or suffers from recurrent glioblastoma.
74. Use of embodiments 51-73, wherein said subject is newly diagnosed with glioblastoma and has a positive methylated O-6-methylguanine-DNA methyltransferase promoter status, wherein said radiopharmaceutical compound is administered to said subject in combination with radiotherapy and an alkylating agent, preferably temozolomide, wherein a first dose of said radiopharmaceutical compound is administered preferably 7 to 10 days prior to initiation of the radiotherapy.
75. Use of embodiments 51-74, wherein said subject is newly diagnosed with glioblastoma and has a negative methylated O-6-methylguanine-DNA methyltransferase promoter status, wherein said radiopharmaceutical compound is administered to said subject in combination with radiotherapy but not in combination with other chemotherapeutic agent, such as temozolomide; and wherein the treatment interval between two administrations of said radiopharmaceutical compound is for the first two intervals 4 weeks, and for the third and any following intervals, 3 weeks; wherein a first dose of said radiopharmaceutical compound is administered preferably 7 to 10 days prior to initiation of the radiotherapy.
76. A method of treating glioblastoma in a subject in need thereof comprising administering to said subject an efficient amount of a radiopharmaceutical compound, wherein said radiopharmaceutical compound is a compound of formula:

M-C—S—P wherein:
M is a radionuclide;
C is a chelating agent capable of chelating said radionuclide;
S is an optional spacer covalently linked between C and P;
P is a somatostatin receptor binding peptide covalently linked to C, either directly or indirectly via S,
wherein said method does not include a concomitant step of irradiating the subject with an efficient dose of ionizing radiations.
77. The method of embodiment 76, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
78. The method of embodiment 76 or 77, wherein C is selected from DOTA (tetraxetan), trizoxetan, DTPA, NTA, EDTA, DO3A, TETA, NOTA, NOTAGA, NODOGA, NODASA, NODAPA, and AAZTA (e.g. AAZTA5) chelating agent, preferably is DOTA, NOTA or DTPA chelating agent, and more preferably is DOTA chelating agent.
79. The method of embodiments 76-78, wherein P is selected from octreotide, octreotate, satoreotide lanreotide, vapreotide, and pasireotide, preferably selected from octreotide and octreotate.
80. The method of embodiments 76-79, wherein the radiopharmaceutical compound is selected from DOTA-OC, DOTA-TOC (edotreotide), satoreotide tetraxetan, DOTA-NOC, DOTA-TATE (oxodotreotide), DOTA-LAN, and DOTA-VAP, preferably selected from DOTA-TOC and DOTA-TATE, more preferably is DOTA-TATE.
81. The method of embodiments 76-81, wherein the radiopharmaceutical compound is [¹⁷⁷Lu]Lu-DOTA-TOC (¹⁷⁷Lu-edotreotide) or [¹⁷⁷Lu]Lu-DOTA-TATE (¹⁷⁷Lu-oxodotreotide), more preferably [¹⁷⁷Lu]Lu-DOTA-TATE (¹⁷⁷Lu-oxodotreotide).
82. The method of embodiments 76-81, wherein said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).
83. The method of embodiments 76-82, wherein said radiopharmaceutical compound is administered 1 to 8 times, preferably 2 to 7 times, more preferably 4 to 6 times, wherein there is a treatment interval between every two administrations of said radiopharmaceutical compound.
84. The method of embodiments 76-83, wherein the administration of said radiopharmaceutical compound comprises 2 to 7 cycles of treatment, with a treatment interval of 2 weeks, or 3 weeks, or 4 weeks, or 5 weeks or even 6 weeks, preferably every 3 weeks.
85. The method of embodiments 76-84, wherein said subject has been selected for the treatment by SPECT/CT or PET/CT or SPECT/MRI, PET/MRI imaging with the same radiopharmaceutical compound as defined for the treatment but with a radiometal suitable for imaging instead of ¹⁷⁷Lu, preferably ⁶⁸Ga, ⁶⁷Ga or ⁶⁴Cu, more preferably ⁶⁸Ga, by evaluating said radiopharmaceutical compound suitable for imaging uptake in said subject.
86. The method of embodiments 76-85, wherein said subject is newly diagnosed with glioblastoma or suffers from recurrent glioblastoma, in particular said subject suffers from recurrent glioblastoma.
87. The method of embodiments 76-86, wherein said subject suffers from recurrent gliolastoma, wherein said wherein said method does not include a concomitant step of administering an alkylating agent, for example temozolomide.
88. The method of embodiments 76-87, wherein said subject suffers from recurrent gliolastoma, said method does not include a concomitant step of irradiating the subject with an efficient dose of ionizing radiations, and does not include a concomitant step of administering an alkylating agent such as temozolomide, wherein said method comprises 2-7 cycles of treatment with said radiopharmaceutical and the treatment interval between two administrations of said radiopharmaceutical compound is 3 weeks.

Embodiments 76-88 can alternatively also expressed in the following formats:

A radiopharmaceutical compound for use in treating glioblastoma in a subject in need thereof wherein a therapeutically efficient amount of said radiopharmaceutical compound is administered to said subject etc.

Use of radiopharmaceutical compound in the preparation of a drug for use in treating glioblastoma in a subject in need thereof etc.

### DETAILED DESCRIPTION

The present disclosure relates to a method for treating glioblastoma in a subject in need thereof by administering a therapeutically efficient amount of a radiopharmaceutical compound to said subject in combination with radiotherapy, and optionally, an alkylating agent, preferably temozolomide.

### General Definitions

The use of the articles “a”, “an”, and “the” in both the description and claims are to be construed to cover both the singular and the plural, unless otherwise indicated herein or clearly contradicted by context. The terms “comprising”, “having”, “being of” as in e.g., a complex “of a radionuclide and a cell receptor binding organic moiety linked to a chelating agent”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” or “ca.” has herein the meaning that the following value may vary for ±20%, preferably ±10%, more preferably ±5%, even more preferably ±2%, even more preferably ±1%.

Unless otherwise defined, “%” has herein the meaning of weight percent (wt %), also referred to as weight by weight percent (w/w %).

“total concentration” refers to the sum of one or more individual concentrations.

“aqueous solution” refers to a solution of one or more solute in water.

The phrase “treatment of” and “treating” includes the amelioration or cessation of a disease, disorder, or a symptom thereof. In particular, with reference to the treatment of a tumor, the term “treatment” may refer to the inhibition of the growth of the tumor, or the reduction of the size of the tumor.

As used herein “glioblastoma” refers to an aggressive brain tumor belonging to Grade IV astrocytoma brain tumor. The term glioblastoma also includes its variants gliosarcoma, giant cell glioblastoma and small cell glioblastoma. Because cells in this tumor vary in size and shape, i.e. they are pleomorphic, glioblastoma is also called glioblastoma multiforme (GBM).

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MRI” stands for magnetic resonance imaging.

As used herein, “CT” stands for computed tomography.

As used herein, the terms “efficient amount” or “therapeutically efficient amount” of a compound refer to an amount of the compound that will elicit the biological or medical response of a subject, for example, ameliorate the symptoms, alleviate conditions, slow or delay disease progression, or prevent a disease.

The terms “patient” and “subject” which are used interchangeably refer to a human being, including for example a subject that has cancer.

“for commercial use” refers to the drug product, e.g. a pharmaceutical aqueous solution, is able to obtain (preferably has obtained) marketing authorization by health authorities, e.g. US-FDA or EMA, by complying with all drug product quality and stability requirements as demanded by such health authorities, is able to be manufactured (preferably is manufactured) from or at a pharmaceutical production site at commercial scale followed by a quality control testing procedure, and is able to be supplied (preferably is supplied) to remotely located end users, e.g. hospitals or patients.

“Combination” refers to either a fixed combination in one dosage unit form, or a combined administration where a compound of the present disclosure and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. the radiolabelled somatostatin binding receptor compound and a combination partner, e.g. the alkylating agent, are both administered to a patient simultaneously in the form of a single entity or dosage. The term “non-fixed combination” means that the therapeutic agents, e.g. the radiolabelled somatostatin binding receptor compound and the combination partner, e.g. the alkylating agent, are both administered to a patient as separate entities either simultaneously, concomittantly or sequentially with no specific time limits, wherein such administration provides therapeutically efficient levels of the two compounds in the body of the patient. The latter also applies to cocktail therapy, e.g. the administration of three or more therapeutic agents.

### The Radiopharmaceutical Compound in the Treatment Methods of the Disclosure

As used herein the term “radiopharmaceutical” refers to a pharmaceutical compound which is labelled with a radionuclide element, typically of metallic nature. Accordingly, the radiopharmaceutical compound is a SSTR binding compound which comprises a radionuclide and which has specific binding affinity to SSTR, for example at least SSTR2 receptor.

Accordingly, a radiolabelled somatostatin receptor binding compound is a compound which comprises a radionuclide and which has specific binding affinity to somatostatin receptor. In some embodiments of the disclosure, said radiolabelled somatostatin receptor binding compound with specific binding affinity to at least SSTR2 receptor.

In these and other embodiments of the disclosure, said radiopharmaceutical compound is a compound of formula

M-C—S—P wherein:
M is a radionuclide;
C is a chelating agent capable of chelating said radionuclide;
S is an optional spacer covalently linked between C and P;
P is a somatostatin receptor binding peptide covalently linked to C, for example via its N-terminal end, either directly or indirectly via S.

Such radiopharmaceutical compound may be selected from octreotide, octreotate, lanreotide, vapreotide, and pasireotide, preferably selected from octreotide and octreotate.

In some embodiments of the disclosure, the radionuclide M is selected radionuclide isotope suitable for PRRT.

Examples of such suitable radionuclide M includes without limitation ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.

As used herein, the term “chelating agent” refers to an organic moiety comprising functional groups that are able to form non-covalent bonds with the radionuclide and, thereby, form stable radionuclide complex. The chelating agent in the context of the present disclosure may be 1,4,7,10-Tetraazacyclododecane-1,4,7,10-tetraacetic acid (DOTA), diethylentriaminepentaacetic acid (DTPA), nitrilotriacetic acid (NTA), ethylenediaminetetraacetic acid (EDTA), 1,4,7,10-tetraazacyclododecane-1,4,7-triacetic acid (DO3A), triethylenetetramine TETA, 1,4,7-triazacyclononane-1,4,7-triacetic acid (NOTA). In many embodiments of the disclosure, the chelating agent is DOTA.

Such chelating agents are either directly linked to the somatostatin receptor binding peptide or connected via a linker molecule, preferably it is directly linked. The linking bond(s) is (are) either covalent or non-covalent bond(s) between the cell receptor binding organic moiety (and the linker) and the chelating agent, preferably the bond(s) is (are) covalent.

As used herein, the term “somatostatin receptor binding peptide” refers to a peptidic moiety with specific binding affinity to somatostatin receptor. Such somatostatin receptor binding peptide may be selected from octreotide, octreotate, lanreotide, vapreotide, and pasireotide, preferably selected from octreotide and octreotate.

According to many embodiments of the methods of the present disclosure, the somatostatin receptor binding peptide linked to the chelating agent is selected from DOTA-OC, DOTA-TOC (edotreotide), DOTA-NOC, DOTA-TATE (oxodotreotide), DOTA-LAN, and DOTA-VAP. In many of these embodiments, the somatostatin receptor binding peptide is DOTA-TOC or

DOTA-TATE. In many such embodiments, the somatostatin receptor binding peptide is DOTA-TATE.

In an embodiment, the radiopharmaceutical compound of the disclosure is ¹⁷⁷Lu-DOTA-TOC (¹⁷⁷Lu-edotreotide) or ¹⁷⁷Lu-DOTA-TATE (¹⁷⁷Lu-oxodotreotide), more preferably ¹⁷⁷Lu-DOTA-TATE (¹⁷⁷Lu-oxodotreotide).

Many embodiments of the disclosure encompass combination therapy with said radiopharmaceutical compound.

The radiopharmaceutical compound is for use in treating glioblastoma in a subject in need thereof wherein a therapeutically efficient amount of said radiopharmaceutical compound is administered to said subject.

In an embodiment, said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).

In another embodiment, the radiopharmaceutical compound for use is administered 1 to 8 times per treatment at the induction phase, preferably 2 to 7 times per treatment, more preferably 4 to 6 times per treatment. The administration of the radiopharmaceutical compound for use may comprises a treatment interval of 2 weeks, or 3 weeks, or 4 weeks, or 5 weeks or even 6 weeks, preferably 3 or 4 weeks, more preferably every 3 weeks.

Accordingly, the cell receptor binding moiety and the chelating agent may form together the following molecules:
DOTA-OC: [DOTA⁰,D-Phe¹]octreotide,
DOTA-TOC: [DOTA⁰,D-Phe¹,Tyr³]octreotide, edotreotide (INN),
represented by the following formulas:

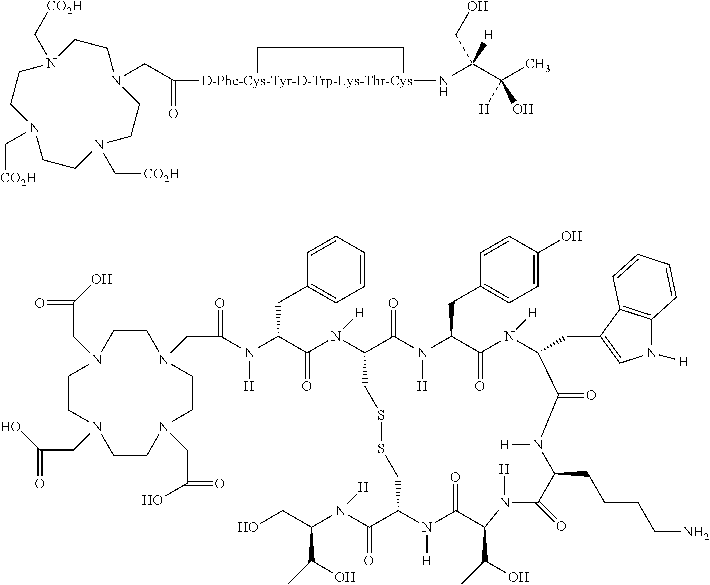

DOTA-NOC: [DOTA⁰, D-Phe¹, 1-Nal³]octreotide, DOTA-TATE: [DOTA⁰,D-Phe¹,Tyr³]octreotate, DOTA-Tyr³-Octreotate, DOTA-d-Phe-Cys-Tyr-d-Trp-Lys-Thr-Cys-Thr (cyclo 2,7), oxodotreotide (INN), represented by the following formula:

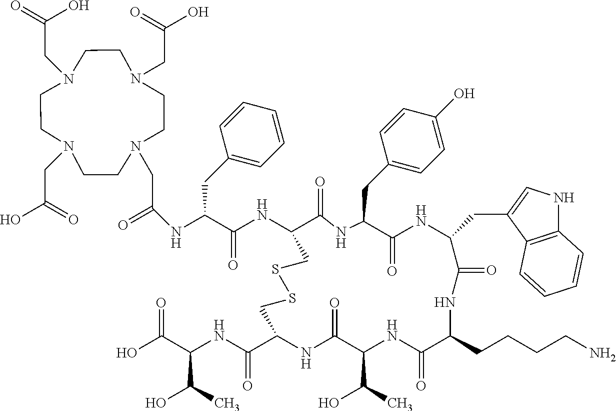 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

DOTA-LAN: [DOTA⁰,D-β-Nal¹]lanreotide,
DOTA-VAP: [DOTA⁰,D-Phe¹,Tyr³]vapreotide.

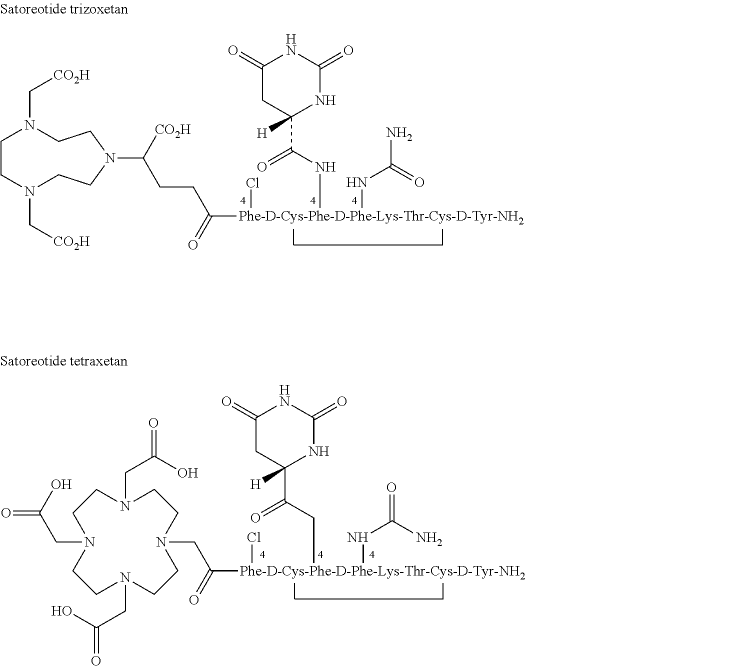

Common “cell receptor binding moiety linked to the chelating agent” molecules of the disclosure for use in the combination therapy are DOTA-TOC, DOTA-TATE, and Satoreotide tetraxetan, more preferably the molecule is DOTA-TATE.

More specifically, in many embodiments of the disclosure, the complex formed by the radionuclide and the cell receptor binding moiety linked to the chelating agent according to the present invention is ¹⁷⁷Lu-DOTA-TATE, which is also referred to as Lutetium (177Lu) oxodotreotide (INN), i.e. hydrogen [N-{[4,7,10-tris(carboxylato-κO-methyl)-1,4,7,10-tetraazacyclododecan-1-yl-κ⁴N¹,N⁴,N⁷,N¹⁰]acetyl-κO}-D-phenylalanyl-L-cysteinyl-tyrosyl-D-tryptophyl-L-lysyl-L-threonyl-L-cysteinyl-L-threoninato cyclic (2→7)-disulfide (4-)](177Lu)lutetate (1-)

and is represented by the following formulas:

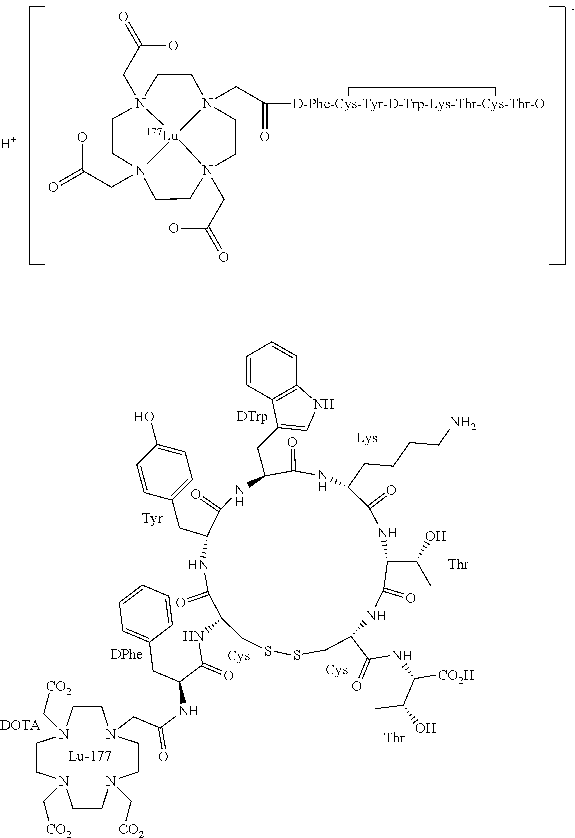

Said radiolabelled somatostatin receptor binding compound is typically formulated for administration of a therapeutically efficient amount in the subject in need thereof.

The radiolabelled somatostatin receptor binding compound can be present in a concentration providing a volumetric radioactivity of 100 MBq/mL or higher. In many embodiments of the disclosure, the volumetric radioactivity is 250 MBq/mL or higher.

In many embodiments of the disclosure, the radiolabeled somatostatin receptor binding compound can be present in a concentration providing a volumetric radioactivity comprised between 100 MBq/mL and 1000 MBq/mL, including between 250 MBq/mL and 500 MBq/mL, for example, at a concentration of about 370 MBq/mL (10 mCi/mL).

The pharmaceutically acceptable excipient can be any of those conventionally used, and is limited only by physico-chemical considerations, such as solubility and lack of reactivity with the active compound(s).

In particular, the one or more pharmaceutically acceptable excipient(s) can be selected from numerous different classes of such pharmaceutically acceptable excipients. Examples of such classes include stabilizers against radiolytic degradation, buffers, sequestering agents and mixtures thereof.

As used herein, “stabilizer against radiolytic degradation” refers to stabilizing agent which protects organic molecules against radiolytic degradation, e.g. when a gamma ray emitted from the radionuclide is cleaving a bond between the atoms of an organic molecules and radicals are forms, those radicals are then scavenged by the stabilizer which avoids the radicals undergo any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”.

As used herein, “sequestering agent” refers to a chelating agent suitable to complex free radionuclide metal ions in the formulation (which are not complexed with the radiolabelled peptide).

Buffers include acetate buffer, citrate buffer and phosphate buffer.

According to many embodiments of the disclosure, the pharmaceutical composition is an aqueous solution, for example an injectable formulation. According to a particular embodiment, the pharmaceutical composition is a solution for infusion.

The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and SHP Handbook on Injectable Drugs, Trissel, 15th ed., pages 622-630 (2009)).

The following clauses refer to various embodiments of suitable pharmaceutical aqueous solution for use in the combination methods of the present disclosure. The following clauses provided are non-limiting.
82. A pharmaceutical aqueous solution comprising
   (a) a complex formed by
      (ai) a radionuclide, and
      (aii) a cell receptor binding organic moiety linked to a chelating agent; and
   (b) at least one stabilizer against radiolytic degradation;
   wherein
   said radionuclide is present in a concentration that it provides a volumetric radioactivity of at least 100 MBq/mL, preferably of at least 250 MBq/mL.
83. The pharmaceutical aqueous solution according to embodiment 82, wherein said stabilizer(s), component (b), is (are) present in a total concentration of at least 0.2 mg/mL, preferably at least 0.5 mg/mL, more preferably at least 1.0 mg/mL, even more preferably at least 2.7 mg/mL.
84. The pharmaceutical aqueous solution according to any one of the preceding embodiments, wherein said radionuclide is present in a concentration that it provides a volumetric radioactivity of from 100 to 1000 MBq/mL, preferably from 250 to 500 MBq/mL.
85. The pharmaceutical aqueous solution according to any one of the preceding embodiments, wherein said stabilizer(s) is (are) present in a total concentration of from 0.2 to 20.0 mg/mL, preferably from 0.5 to 10.0 mg/mL, more preferably from 1.0 to 5.0 mg/mL, even more preferably from 2.7 to 4.1 mg/mL.
86. The pharmaceutical aqueous solution according to any one of the preceding embodiments, wherein the component (b) is only one stabilizers against radiolytic degradation, i.e. only a first stabilizer.
87. The pharmaceutical aqueous solution according to any one of the preceding embodiments, wherein the component (b) are at least two stabilizers against radiolytic degradation, i.e. at least a first and a second stabilizer, preferably only two stabilizers, i.e. only a first and a second stabilizer.
88. The pharmaceutical aqueous solution according to any one of the embodiments 86 to 87, wherein the first stabilizer is present in a concentration of from 0.2 to 5 mg/mL, preferably from 0.5 to 5 mg/mL, more preferably from 0.5 to 2 mg/mL, even more preferably from 0.5 to 1 mg/mL, even more preferably from 0.5 to 0.7 mg/mL.
89. The pharmaceutical aqueous solution according to embodiment 87 or 88, wherein the second stabilizer is present in a concentration of from 0.5 to 10 mg/mL, more preferably from 1.0 to 8.0 mg/mL, even more preferably from 2.0 to 5.0 mg/mL, even more preferably from 2.2 to 3.4 mg/mL.
90. The pharmaceutical aqueous solution according to any one of the preceding embodiments, wherein the stabilizer(s) is (are) selected from gentisic acid (2,5-dihydroxybenzoic acid) or salts thereof, ascorbic acid (L-ascorbic acid, vitamin C) or salts thereof (e.g. sodium ascorbate), methionine, histidine, melatonin, ethanol, and Se-methionine, preferably selected from gentisic acid or salts thereof and ascorbic acid or salts thereof.
97. The pharmaceutical aqueous solution according to any one of the preceding embodiments, which is free of ethanol.
98. The pharmaceutical aqueous solution according to any one of the embodiments 86 to 90, wherein the first stabilizer is selected from gentisic acid and ascorbic acid, preferably the first stabilizer is gentisic acid.
99. The pharmaceutical aqueous solution according to any one of the embodiments 87 to 91, wherein the second stabilizer is selected from gentisic acid and ascorbic acid, preferably the second stabilizer is ascorbic acid.
100. The pharmaceutical aqueous solution according to any one of the embodiments 87 to 89, wherein the first stabilizer is gentisic acid or a salt thereof and the second stabilizer is ascorbic acid or a salt thereof, and the ratio of the concentration (in mg/ml) of the first stabilizer to the concentration (in mg/mL) of the second stabilizer is from 1:3 to 1:7, preferably from 1:4 to 1:5.
101. The pharmaceutical aqueous solution according to any one of the preceding embodiments, wherein the radionuclide is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.
102. The pharmaceutical aqueous solution according to any one of the preceding embodiments, wherein the cell receptor binding moiety is a somatostatin receptor binding peptide, preferably said somatostatin receptor binding peptide is selected from octreotide, octreotate, lanreotide, vapreotide and pasireotide, preferably selected from octreotide and octreotate.
103. The pharmaceutical aqueous solution according to any one of the preceding embodiments, wherein the chelating agent is selected from DOTA, DTPA, NTA, EDTA, DO3A, TETA and NOTA, preferably is DOTA.
104. The pharmaceutical aqueous solution according to any one of the preceding embodiments, wherein the cell receptor binding moiety and the chelating agent form together molecules selected from DOTA-OC, DOTA-TOC (edotreotide), DOTA-NOC, DOTA-TATE (oxodotreotide), DOTA-LAN, and DOTA-VAP, preferably selected from DOTA-TOC and DOTA-TATE, more preferably is DOTA-TATE.
105. The pharmaceutical aqueous solution according to any one of the preceding embodiments, wherein the radionuclide, the cell receptor binding moiety and the chelating agent form together the complex ¹⁷⁷Lu-DOTA-TOC (¹⁷⁷Lu-edotreotide) or ¹⁷⁷Lu-DOTA-TATE (¹⁷⁷Lu-oxodotreotide), preferably ¹⁷⁷Lu-DOTA-TATE.
106. The pharmaceutical aqueous solution according to any one of the preceding embodiments, further comprising a buffer, preferably said buffer is an acetate buffer, preferably in an amount to result in a concentration of from 0.3 to 0.7 mg/ml (preferably about 0.48 mg/mL) acetic acid and from 0.4 to 0.9 mg/ml (preferably about 0.66 mg/mL) sodium acetate.
107. The pharmaceutical aqueous solution according to any one of the preceding embodiments, further comprising a sequestering agent, preferably said sequestering agent is diethylentriaminepentaacetic acid (DTPA) or a salt thereof, preferably in an amount to result in a concentration of from 0.01 to 0.10 mg/mL (preferably about 0.05 mg/mL).
108. The pharmaceutical aqueous solution according to any one of the preceding embodiments, which has a shelf life of at least 24 hours (h) at ≤25° C., at least 48 h at ≤ 25° C., at least 72 h at ≤25° C., of from 24 h to 120 h at ≤25° C., from 24 h to 96 h at ≤25° C., from 24 h to 84 h at $25° C., from 24 h to 72 h at ≤25° C., in particular has a shelf life of 72 h at ≤25° C.
109. The pharmaceutical aqueous solution according to any one of the preceding embodiments, wherein said solution is produced at commercial scale manufacturing, in particular is produced at a batch size of at least 20 GBq, at least 50 GBq, or at least 70 GBq.
110. The pharmaceutical aqueous solution according to any one of the preceding embodiments, which is ready-to-use.
111. The pharmaceutical aqueous solution according to any one of the preceding embodiments, which is for commercial use.
112. A pharmaceutical aqueous solution, comprising
   (a) a complex formed by
      (ai) the radionuclide 177-Lutetium (¹⁷⁷Lu), present in a concentration that it provides a volumetric radioactivity of from 250 to 500 MBq/mL, and
      (aii) the chelating agent linked somatostatin receptor binging organic moiety DOTA-TATE (oxodotreotide) or DOTA-TOC (edotreotide);
   (bi) gentisic acid or a salt thereof as the first stabilizer against radiolytic degradation present in a concentration of from 0.5 to 1 mg/ml;
   (bii) ascorbic acid or a salt thereof as the second stabilizer against radiolytic degradation present in a concentration of from 2.0 to 5.0 mg/mL.
113. The pharmaceutical aqueous solution according to embodiment 109, further comprising:
   (c) Diethylentriaminepentaacetic acid (DTPA) or a salt thereof in a concentration of from 0.01 to 0.10 mg/mL.
114. The pharmaceutical aqueous solution according to embodiments 109 or 110, further comprising:
   (d) acetic acid in a concentration of from 0.3 to 0.7 mg/ml and sodium acetate in a concentration from 0.4 to 0.9 mg/mL.
115. The pharmaceutical aqueous solution according to any one of the preceding embodiments wherein the stabilizer(s) is (are) present in the solution during the complex formation of components (ai) and (aii).
116. The pharmaceutical aqueous solution according to any one of embodiments 86 to 115 wherein only the first stabilizer is present during the complex formation of components (ai) and (aii), preferably in an amount to result in a concentration of from 0.5 to 5 mg/mL, more preferably from 0.5 to 2 mg/mL, even more preferably from 0.5 to 1 mg/mL, even more preferably from 0.5 to 0.7 mg/mL, in the final solution.
117. The pharmaceutical aqueous solution according to any one of embodiments 86 to 116 wherein a part of the amount of the second stabilizer is already present in the solution during the complex formation of components (ai) and (aii) and another part of the amount of the second stabilizer is added after the complex formation of components (ai) and (aii).
118. The pharmaceutical aqueous solution according to any one of embodiments 86 to 117 wherein the second stabilizer is added after the complex formation of components (ai) and (aii).
119. The pharmaceutical aqueous solution according to embodiment 87 or 118 wherein the second stabilizer is added after the complex formation of components (ai) and (aii), preferably in an amount to result in a concentration of from 0.5 to 10 mg/mL, more preferably from 1.0 to 8.0 mg/mL, even more preferably from 2.0 to 5.0 mg/mL, even more preferably from 2.2 to 3.4 mg/mL, in the final solution.
120. The pharmaceutical aqueous solution according to any one of the preceding embodiments, further comprising a sequestering agent, added after the complex formation of components (ai) and (aii), for removing any uncomplexed Lu, preferably said sequestering agent is diethylentriaminepentaacetic acid (DTPA) or a salt thereof, preferably in an amount to result in a concentration of from 0.01 to 0.10 mg/mL (preferably about 0.05 mg/mL) in the final solution.

Often, a solution for infusion of ¹⁷⁷Lu-DOTA-TATE or ¹⁷⁷Lu-DOTA-TOC such as one with specific activity concentration of 370 MBq/mL (±5%) is used in the combination methods of the present disclosure.

A particular process for manufacturing the pharmaceutical aqueous solution as defined in any one of the preceding embodiments, may comprise the process steps:
(1) Forming a complex of the radionuclide and the chelating agent linked cell receptor binding organic moiety by
   (1.1) preparing an aqueous solution comprising the radionuclide;
   (1.2) preparing an aqueous solution comprising the chelating agent linked cell receptor binding organic moiety, a first stabilizer, optionally a second stabilizer; and
   (1.3) mixing the solutions obtained in steps (1.1) and (1.2) and heating the resulting mixture;
(2) Diluting the complex solution obtained by step (1) by
   (2.1) preparing an aqueous dilution solution optionally comprising a second stabilizer; and
   (2.2.) mixing the complex solution obtained by step (1) with the dilution solution obtained by the step (2.1).

### Radiotherapy as Used in the Combination Therapy

In an embodiment, the method of treating glioblastoma in a subject in need thereof includes a step of irradiating the subject with an efficient dose of ionizing radiations i.e. radiotherapy.

As used herein, the term “radiotherapy” is used for the treatment of diseases of oncological nature with irradiation corresponding to ionizing radiation. Ionizing radiation deposits energy that injures or destroys cells in the area being treated (the target tissue) by damaging their genetic material, making it impossible for these cells to continue to grow.

In specific embodiment, the method of the disclosure comprises exposing the tumor to be treated to an efficient dose of ionizing radiations, wherein said ionizing radiations are photons, e.g. X-rays. Depending on the amount of energy they possess, the rays can be used to destroy cancer cells on the surface of or deeper in the body. The higher the energy of the X-ray beam, the deeper the X-rays can go into the target tissue. Linear accelerators and betatrons produce X-rays of increasingly greater energy. The use of machines to focus radiation (such as X-rays) on a cancer site is called external beam radiotherapy.

In an alternative embodiment of the method of treatment according to the disclosure, gamma rays are used. Gamma rays are produced spontaneously as certain elements (such as radium, uranium, and cobalt 60) release radiation as they decompose, or decay.

Ionizing radiations are typically of 2 keV to 25000 keV, in particular of 2 keV to 6000 keV (i.e. 6 MeV) or of 2 keV to 1500 keV (such as cobalt 60 source).

A person of ordinary skill in the radiotherapy art knows how to determine an appropriate dosing and application schedule, depending on the nature of the disease and the constitution of the patient. In particular, the person knows how to assess dose-limiting toxicity (DLT) and how to determine the maximum tolerated dose (MTD) accordingly.

The amount of radiation used in radiation therapy is measured in gray (Gy), and varies depending on the type and stage of cancer being treated. For curative cases, the typical total dose for a solid tumor ranges from 20 to 120 Gy. Many other factors are considered by radiation oncologists when selecting a dose, including whether the patient is receiving chemotherapy, patient co-morbidities, whether radiation therapy is being administered before or after surgery, and the degree of success of surgery.

The total dose is typically fractionated (spread out over time). Amount and schedules (planning and delivery of ionizing radiations, fraction dose, fraction delivery schema, total dose alone or in combination with other anti-cancer agents etc) is defined for any disease/anatomical site/disease stage patient setting/age and constitutes the standard of care for any specific situation.

A typical conventional fractionation schedule for adults for the methods of the present disclosure may be 1 to 4 Gy per day, preferably around 2 Gy/day during a period between 3 to 7 days, preferably around 5 days per week during a period between 4 to 8 weeks, preferably 6 weeks. In specific embodiments, said radiotherapy consists of exposing the subject to a total dose of ionizing radiations between 50 and 70 Gy, for example 60 Gy.

In other specific embodiments, the subject is exposed to a dose of ionizing radiations per fraction of about 2 to 12 Gy, and the total dose is administered preferably in a maximum of 6 fractions. In another words, said radiotherapy is conducted for 5 consecutive days followed by 2 days of rest for 6 consecutive weeks.

In specific embodiment where the subject is suffering from glioblastoma, the radiation therapy applied of the herein disclosed methods is a whole-brain radiotherapy (WBRT).

### Alkylating Agent as Used in the Combination Therapy

The method of treating glioblastoma in a subject in need thereof optionally includes a step of administering the radiopharmaceutical compound to said subject in combination with radiotherapy and with a therapeutically efficient amount of an alkylating agent, preferably temozolomide.

Alkylating agents are divided into different classes, including:
Nitrogen mustards: such as mechlorethamine (nitrogen mustard), chlorambucil, cyclophosphamide (Cytoxan®), ifosfamide, and melphalan;
Nitrosoureas: such as streptozocin, carmustine (BCNU), and lomustine;
Alkyl sulfonates: busulfan;
Triazines: dacarbazine (DTIC) and temozolomide (Temodar®); and
Ethylenimines: thiotepa and altretamine (hexamethylmelamine).

As used herein, “temozolomide” refers to an triazines alkylating agent and more specifically compound of formula 3,4-Dihydro-3-methyl-4-oxoimidazo[5,1-d][1,2,3,5]tetrazine-8-carboxamide and pharmaceutically acceptable salts thereof (CAS number of 85622-93-1). Alkylating agents directly damage DNA (the genetic material in each cell) to keep the cell from reproducing. These drugs work in all phases of the cell cycle and are used to treat many different cancers, including glioblastoma, leukemia, lymphoma, Hodgkin disease, multiple myeloma, and sarcoma, as well as cancers of the lung, breast, and ovary.

In an embodiment, said alkylating agent, preferably temozolomide, is administered at an induction phase at a dose of between 50 to 100 mg/m²/day, preferably around 75 mg/m²/day each day for a period between 4 to 8 weeks, preferably 6 weeks.

As used herein, “induction phase” refers to the period in which said alkylating agent, preferably temozolomide, is first administered to the subject wherein the period has a duration of up to 11 weeks, for example from week 1 day 1 to end of week 11 day 7.

In an embodiment, both radiotherapy and alkylating agent, preferably temozolomide, are initiated the same day. In certain aspect the alkylating agent, preferably temozolomide, is concomittantly administered with the radiotherapy without interruption.

In certain embodiment, said alkylating agent, preferably temozolomide, is daily administered at a first dose during the concomitant administration with the radiotherapy, for example for a period of 6 weeks, and at a second dose during a maintenance phase, following the concomitant administration with the radiotherapy for example, for a period up to 24 weeks, wherein said second dose is at least twice the first dose radiopharmaceutical compound.

As used herein, “maintenance phase” refers to the period starting after the induction phase or the concomitant administration with the radiotherapy, with an increased dose as compared to dose at the induction phase, for example at week 12 day 1 with a duration of up to 25 weeks.

In specific embodiments, in this maintenance phase, said alkylating agent, preferably temozolomide, is administered at a dose of between 50 to 400 mg/m²/day, preferably between 75 to 300 mg/m²/day, more preferably between 150 to 200 mg/m²/day each day for 5 consecutive days followed by 2 days of rest every 28 days for a period between 20 to 28 weeks, preferably 24 weeks.

In an embodiment, the alkylating agent, preferably temozolomide, is formulated for oral administration.

### The Combination Therapy

In a specific embodiment, the method of treating glioblastoma in a subject in need thereof comprises administering to said subject an efficient amount of a radiopharmaceutical compound, preferably [¹⁷⁷Lu]Lu-DOTA-TATE (¹⁷⁷Lu-oxodotreotide).

In another embodiment, the present disclosure is directed to methods of treating glioblastoma in a subject in need thereof comprising administering to said subject an efficient amount of a radiopharmaceutical compound in combination with a step of irradiating the subject with an efficient dose of ionizing radiations, and optionally, with a therapeutically efficient amount of an alkylating agent, preferably temozolomide.

The disclosure thus relates to a radiopharmaceutical compound for use in treating glioblastoma in a subject in need thereof, a therapeutically efficient amount of said radiopharmaceutical compound is administered to said subject in combination, simultaneously, separately or sequentially, with radiotherapy, and optionally, with a therapeutically efficient amount of an alkylating agent, preferably temozolomide.

The disclosure also relates to the use of radiopharmaceutical compound in the preparation of a drug for use in treating glioblastoma in a subject in need thereof wherein a therapeutically efficient amount of said radiopharmaceutical compound is administered to said subject in combination, simultaneously, separately or sequentially, with radiotherapy, and optionally, with a therapeutically efficient amount of an alkylating agent, preferably temozolomide.

In various embodiments of the disclosure, the combination therapy comprises jointly (i) administering to a subject in need thereof therapeutically efficient amounts of a pharmaceutical composition comprising a radiopharmaceutical compound; and (ii) irradiating the subject with an efficient dose of ionizing radiations, and optionally, (iii) administering to a subject in need thereof therapeutically efficient amounts of a pharmaceutical composition comprising an alkylating agent, preferably temozolomide.

As used herein, the term “jointly” means that the therapeutic agents may be given separately (in a chronologically staggered manner, especially a sequence-specific manner) in such time intervals to show a (preferably synergistic) interaction (i.e. joint therapeutic effect).

In various embodiments of the disclosure, a combined administration where the radiopharmaceutical compound (e.g. [¹⁷⁷Lu]Lu-DOTA-TATE) and the radiotherapy is administered independently at the same time or separately within time intervals, especially where these time intervals allow that the combination partners show a cooperative, e.g. synergistic effect.

In an embodiment, the radiopharmaceutical compound is first administered 1 to 20 days, preferably 3 to 15 days, more preferably 7 to 10 days prior to initiation of radiotherapy

Administration of the radiopharmaceutical compound may comprises a treatment interval of 2 weeks, or 3 weeks, or 4 weeks, or 5 weeks or even 6 weeks, preferably 3 or 4 weeks, more preferably every 3 weeks.

Advantageously, the combined effect of the radiopharmaceutical compound and radiotherapy therapies increases the overall response rate to at least 10%, 20%, 30%, 40%, or at least 50% as compared to single radiotherapy.

The single components or their precursor, typically non-labelled DOTATE, may be packaged in a kit or separately. One or both of the components (e.g., powders or liquids) may be reconstituted or diluted to a desired dose prior to administration.

In certain aspects, the administration of the composition comprising the radiopharmceutical compound to a subject eligible for said treatment can inhibit, delay, and/or reduce tumor growth in the subject. In certain aspects, the growth of the tumor is delayed by at least 30%, 40%, 50% or 60% in comparison to an untreated control subject. In certain aspects, the growth of the tumor is delayed by at least 60% in comparison to an untreated control subject. In certain aspects, the growth of the tumor is delayed by at least 30%, 40%, 50% or 6% in comparison to the predicted growth of the tumor without the treatment. In certain aspects, the growth of the tumor is delayed by at least 60% in comparison to the predicted growth of the tumor without the treatment.

In certain aspects, the administration of the composition comprising the radiopharmaceutical composition to a subject eligible for said treatment can increase the length of survival of the subject. In certain aspects, the increase in survival is in comparison to an untreated control subject. In certain aspects, the increase in survival is in comparison to the predicted length of survival of the subject without the treatment. In certain aspects, the length of survival is increased by at least 1.1 times, 1.2 times, 1.3 times or 1.4 times the length in comparison to an untreated control subject. In certain aspects, the length of survival is increased by at least 1.2 times the length in comparison to an untreated control subject. In certain aspects, the length of survival is increased by at least 1.1 times, 1.2 times, 1.3 times or 1.4 times the length in comparison to the predicted length of survival of the subject without the treatment. In certain aspects, the length of survival is increased by at least 1.2 times the length in comparison to the predicted length of survival of the subject without the treatment. In certain aspects, the length of survival is increased by at least one month, two months, three months, four months, five months, or six months in comparison to an untreated control subject. In certain aspects, the length of survival is increased by at least three months, or four months in comparison to an untreated control subject. In certain aspects, the length of survival is increased by at least one month, two months, three months, four months, or six months in comparison to the predicted length of survival of the subject without the treatment. In certain aspects, the length of survival is increased by at least three months, or four months in comparison to the predicted length of survival of the subject without the treatment.

### Methods for Selecting a Subject for the Combination Treatment

In certain embodiments of the disclosure, said glioblastoma is SSTR positive disease. In an embodiment, the subject is selected for the treatment by SPECT/CT or PET/CT or SPECT/MRI, PET/MRI imaging with the same radiopharmaceutical compound as defined for the treatment but wherein M is a radiometal suitable for imaging i.e. imaging radiopharmaceutical compound. Typical radiometal suitable for use as contrast agent in imaging include the following: ¹¹¹In, ^{133m}In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁶¹Cu ¹⁷⁷Lu, ⁸⁶Y, ⁵¹Cr, ^{52m}Mn, ¹⁵⁷Gd, ¹⁶⁹Yb, ¹⁷²Tm, ^{117m}Sn, ¹²³I, ¹²⁴I, ¹²⁵I, ¹⁸F, Al¹⁸F, ¹⁵²Tb, ¹⁵⁵Tb, ⁸²Rb, ⁸⁹Zr, ⁴³Sc, ⁴⁴Sc.

According to a preferred embodiment, the radiometal suitable for imaging is ⁶⁷Ga, ⁶⁸Ga or ⁶⁴Cu, preferably ⁶⁸Ga.

In an embodiment, the subject is selected by evaluating the [⁶⁸Ga]Ga-DOTA-TATE uptake by PET/CT or PET/MRI scan at the tumor region, e.g. whole brain.

Thus, the disclosure also relates to methods for determining whether a human patient having glioblastoma can be selected for the combination therapy, said method comprising the steps of:
(i) administering an efficient amount of an imaging radiopharmaceutical compound as a contrast agent for imaging the uptake of said radiopharmaceutical compound,
(ii) acquiring an image by PET/MRI or PET/CT of said patient, and
(iii) comparing with a control image.

The objective of the above method is to select the patient with SSTR-positive tumors, i.e. which patients are good responders to a treatment with the radiopharmaceutical compound of the disclosure. SSTR-positive tumors may be advantageously detected by evaluating the uptake of a imaging radiopharmaceutical compound by PET/MRI or PET/CT imaging after injection of said imaging radiopharmaceutical compound as contrast agent.

As used herein, a good responder is a patient selected from a patient population which shows statistically better response to a treatment as compared to a randomized patient population (i.e. which has not been selected by the selection step of the present method), and/or which shows less side effects to a treatment as compared to a randomized patient population (i.e. which has not been selected by the selection step of the present method).

In certain aspect, the [⁶⁸Ga]Ga-DOTA-TATE is provided in a kit called NETSPOT® (Gallium Ga 68 dotatate (USAN)). This kit is for radiopharmaceutical preparation of [⁶⁸Ga]Ga-DOTA-TATE approved in the United States of America (USA) (2016), Canada (2019) and Switzerland (2019) with the following indication: After radiolabeling with (⁶⁸Ga), is a radioactive diagnostic agent indicated for use with PET for localization of SSTR-positive neuroendocrine tumors (NETs) (NETSPOT® PI).

In an embodiment, the selection of subject is performed between 10 to 18 days, preferably around 14 days prior to the first administration of the radiopharmaceutical compound.

In certain embodiment, said imaging radiopharmaeutical is administered at a dose between 1.5 MBq/kg (0.040 mCi/kg) and 2.5 MBq/kg (0.067 mCi/kg), preferably around 2 MBq/kg of body weight (0.054 mCi/kg), with a minimum dose of 100 MBq (2.7 mCi) and maximum dose of 200 MBq (5.4 mCi), typically by intravenous injection, preferably slow intravenous injection.

Images of subject's body are then acquired by PET/MRI or PET/CT imaging and the images are compared with a control image to identify whether the lesions identified by conventional imaging, for example by MRI, CT, SPECT or PET, are also identified by said imaging radiopharmaceutcal compound uptake, i.e. [⁶⁸Ga]Ga-DOTA-TATE uptake. Typically, PET/MRI or PET/CT imaging is performed between 30 to 120 minutes, preferably between 60 to 90 minutes after the intravenous administration of said imaging radiopharmaceutical compound to the subject.

In a specific embodiment of the method, a subject is selected for the combination therapy of the disclosure fulfils the following condition: at least 10%, preferably more than 20%, preferably more than 30%, preferably more than 40%, preferably more than 50%, preferably more than 60%, preferably more than 70%, preferably more than 80% of the lesions as detected by conventional imaging in said subject, for example by MRI, CT, SPECT or PET, are also identified by the imaging radiopharmaceutical compound uptake, e.g. [⁶⁸Ga]Ga-DOTA-TATE uptake, as determined by PET/MRI or PET/CT imaging in said subject.

In specific embodiment, the term “lesion” refers to measurable tumor lesions according to Modified RANO criteria as defined in Ellingson B M. Wen P Y, Cloughesy T F. Modified Criteria for Radiographic Response Assessment in Glioblastoma Clinical Trials. Neurotherapeutics. 2017 April; 14(2):307-320. doi: 10.1007/s13311-016-0507-6. PMID: 28108885; PMCID: PMC5398984.

In certain aspect, said subject is newly diagnosed with glioblastoma or suffers from recurrent glioblastoma.

In another embodiment, the subject is further selected by evaluating its methylated O-6-methylguanine-DNA methyltransferase (MGMT) promoter methylation status. Typically, subjects receiving alkylating agent, preferably temozolomide, are selected from subjects with positive MGMT promoter status.

Patients harboring methylation at the MGMT promoter in the tumor are those mostly benefiting from alkylating agents such as temozolomide. In certain aspects, in this group of patients with methylated MGMT promoter, the radiopharmaceutical compound of the disclosure may be assessed in combination with concomitant radiotherapy and alkylating agent, preferably temozolomide, followed by the radiopharmaceutical compound in combination with alkylating agent, preferably temozolomide, maintenance.

### EXAMPLES

### Example 1: Clinical Study for Treating Glioblastoma Subjects

Provided herein is a protocol example describing a prospective phase Ib Dose Finding Study Assessing Safety and Activity of [¹⁷⁷Lu]Lu-DOTA-TATE in Newly Diagnosed Glioblastoma in Combination with Radiotherapy with or without Temozolomide and in Recurrent Glioblastoma as Single Agent.

### Synopsis

| | |
|---|---|
| Brief title | A Dose Finding Study of [¹⁷⁷Lu]Lu-DOTA-TATE in Newly Diagnosed |
| | Glioblastoma in Combination with Standard of Care and in Recurrent |
| | Glioblastoma as a Single Agent. |
| Sponsor and | Novartis, Phase Ib |
| Clinical Phase | |
| Investigation type | Other: Peptide Receptor Radionuclide Therapy, [¹⁷⁷Lu]Lu-DOTA-TATE |
| Study type | Interventional |
| Purpose | This study aims to establish the recommended dose of [¹⁷⁷Lu]Lu-DOTA-TATE |
| | in combination with the standard of care or as single agent in three different |
| | groups of participants with Glioblastoma. In addition, this study will |
| | investigate the safety of [⁶⁸Ga]Ga-DOTA-TATE and describe its uptake |
| | characteristics in participants with Glioblastoma. |
| Primary | To determine the recommended dose of [¹⁷⁷Lu]Lu-DOTA-TATE in 3 different |
| Objective(s) | groups of Glioblastoma: |
| | Group 1: Newly diagnosed glioblastoma participants with methylated O-⁶- |
| | methylguanine-DNA methyltransferase (MGMT) promoter treated with |
| | [¹⁷⁷Lu]Lu-DOTA-TATE in combination with concomitant radiotherapy and |
| | temozolomide followed by [¹⁷⁷Lu]Lu-DOTA-TATE and temozolomide in |
| | maintenance. |
| | Group 2: Newly diagnosed glioblastoma participants with unmethylated |
| | MGMT promoter treated with [¹⁷⁷Lu]Lu-DOTA-TATE in combination with |
| | radiotherapy followed by [¹⁷⁷Lu]Lu-DOTA-TATE alone. |
| | Group 3: Recurrent glioblastoma participants treated with [¹⁷⁷Lu]Lu-DOTA- |
| | TATE. |
| Secondary | To assess the safety and tolerability of [¹⁷⁷Lu]Lu-DOTA-TATE in |
| Objectives | combination with radiotherapy + temozolomide or with |
| | radiotherapy in participants with newly diagnosed glioblastoma and as a |
| | single agent in participants with recurrent glioblastoma. |
| | To assess anti-tumor activity of [¹⁷⁷Lu]Lu-DOTA-TATE in combination with |
| | radiotherapy + temozolomide or with radiotherapy in participants |
| | with newly diagnosed glioblastoma or as a single agent in participants with |
| | recurrent glioblastoma using modified response assessment in neuro- |
| | oncology (RANO) criteria. |
| | To assess pharmacokinetics and dosimetry of [¹⁷⁷Lu]Lu-DOTA- |
| | TATE in participants with newly diagnosed or recurrent glioblastoma. |
| | To assess the safety and tolerability of [⁶⁸Ga]Ga-DOTA-TATE in all |
| | participants. |
| Study design | This is a Phase Ib, open-label, multi-center, dose finding study in 3 parallel |
| | groups: eligible participants with newly diagnosed glioblastoma will be |
| | assigned to Group 1 or Group 2 depending on MGMT promoter methylation |
| | status. Eligible participants with recurrent glioblastoma will be assigned to |
| | Group 3. |
| | The study for each participant consists of a Screening period, a Treatment |
| | period and a 12-month Follow-up period. |
| Rationale | There may be benefits of [¹⁷⁷Lu]Lu-DOTA-TATE in patients with glioblastoma. |
| | Despite the therapeutic options currently available, prognosis of glioblastoma |
| | is dismal. In vitro and clinical studies have shown presence of somatostatin |
| | receptor expression in glioblastoma samples, particularly in the tumor |
| | microenvironment, and DOTA tracer uptake in glioblastoma patients. In light of |
| | the data available, glioblastoma could be a plausible target for peptide |
| | receptor radionuclide therapy; specifically, by targeting somatostatin receptor |
| | expressing cells and capability to deliver large irradiation of tumor margins |
| | with “cross fire” effects. Given the significant unmet need in glioblastoma, it is |
| | evident that new therapeutic approaches are needed. Thus [¹⁷⁷Lu]Lu-DOTA- |
| | TATE is a potential therapeutic modality in glioblastoma. |
| Study population | This study will be conducted in 3 separate groups of participants with newly |
| | diagnosed glioblastoma with methylated or unmethylated MGMT promoter or |
| | participants with recurrent glioblastoma. Approximately 15 male and/or female |
| | adult participants will be enrolled in each group. |
| Key Inclusion | Common Criteria: |
| criteria | Participant is ≥18 years on the day of signing informed consent form |
| | Histologically confirmed glioblastoma |
| | Adequate bone marrow, organ function and electrolyte values |
| | Newly Diagnosed Glioblastoma: |
| | Presence of Gadolinium enhancing tumor in pre-surgery magnetic |
| | resonance imaging (MRI) |
| | Karnofsky Performance Score (KPS) ≥70% |
| | Recurrent Glioblastoma: |
| | Participant has experienced first or second recurrence of their |
| | glioblastoma, after standard or experimental therapy that includes prior |
| | radiation therapy |
| | Evidence of recurrent disease demonstrated by disease progression using |
| | modified Response Assessment in Neuro-Oncology (mRANO) criteria |
| | KPS ≥60% |
| | [⁶⁸Ga]Ga-DOTA-TATE uptake by positron emission tomography/computed |
| | tomography (PET/CT) or PET/MRI scan at the tumor region |
| | Presence of Gadolinium enhancement in the tumor region in MRI at the |
| | time of diagnosis of tumor recurrence |
| Key Exclusion | Common Criteria: |
| criteria | Participant is receiving additional, concurrent, active therapy for |
| | glioblastoma outside of the trial |
| | Extensive leptomeningeal disease |
| | History of another active malignancy in the previous 3 years prior to study |
| | entry |
| | Newly Diagnosed Glioblastoma: |
| | Any prior treatment for glioma of any grade |
| | Recurrent Glioblastoma: |
| | Early disease progression prior to 3 months from the completion of |
| | radiotherapy |
| | More than 2 prior lines for systemic therapy |
| | Previous treatment with bevacizumab |
| Study treatment | All participants will be scanned with [⁶⁸Ga]Ga-DOTA-TATE PET/CT (or |
| | PET/MRI) during screening. |
| | Participants in Group 1 (concomitant radiotherapy + temozolomide and |
| | temozolomide maintenance) will receive treatment with [¹⁷⁷Lu]Lu-DOTA- |
| | TATE every 4 weeks +/− 2 days, up to 6 administrations. Radiotherapy |
| | and temozolomide will be administered 7 to 10 days after the first |
| | administration of [¹⁷⁷Lu]Lu-DOTA-TATE. Temozolomide will be |
| | administered orally at a dose of 75 mg/m²/day during the concomitant |
| | period, concurrently with radiotherapy. Radiotherapy will be delivered at a |
| | dose of 2 Gray (Gy)/day, 5 days per week followed by 2 days of rest, for 6 |
| | consecutive weeks with a total dose of 60 Gy (without interruption). During |
| | the maintenance period, there is an intra-patient dose escalation in |
| | temozolomide treatment. The dosage of temozolomide is 150 mg/m² in |
| | Cycle 1 of maintenance period, and then to 200 mg/m² in Cycle 2 and |
| | beyond in the maintenance period, if 150 mg/m² temozolomide treatment |
| | is well tolerated in Cycle 1. |
| | Participants in Group 2 will receive treatment with [¹⁷⁷Lu]Lu-DOTA-TATE |
| | every 4 weeks +/− 2 days for the first 3 doses (during the radiotherapy |
| | period) followed by every 3 weeks +/− 2 days as single agent, up to a total |
| | of 6 administrations. Radiotherapy will be initiated 7 to 10 days after the |
| | first administration of [¹⁷⁷Lu]Lu-DOTA-TATE and will be delivered at a |
| | dose of 2 Gy/day, 5 days per week followed by 2 days of rest, for 6 |
| | consecutive weeks. |
| | Participants in Group 3 will receive [¹⁷⁷Lu]Lu-DOTA-TATE every 3 |
| | weeks +/− 2 days. |
| | [¹⁷⁷Lu]Lu-DOTA-TATE and the kit for the radiopharmaceutical preparation of |
| | [⁶⁸Ga]Ga-DOTA-TATE will be supplied by the Sponsor. The Sponsor will also |
| | provide the 2.5% Lys-Arg amino-acid solution for infusion (if it can't be |
| | compounded locally). Temozolomide and radiotherapy components will be |
| | provided locally. |
| Efficacy | Brain MRI-Gadolinium-enhanced brain MRI 4 weeks after completion of |
| assessments | radiotherapy (Groups 1 + 2) or within 8 days before treatment start (Group |
| | 3) and every 8 weeks until confirmed progression or end of study |
| | [⁶⁸Ga]Ga-DOTA-TATE imaging PET scan for somatostatin receptor |
| | imaging at screening |
| | Whole body planar imaging for dosimetry assessment at Week 5 (Groups |
| | 1 + 2) and Week 4 (Group 3) |
| | Single-photon emission computed tomography (SPECT)/CT imaging for |
| | dosimetry assessment at Week 5 (Groups 1 + 2) and Week 4 (Group 3) |
| Pharmacokinetic | Participants will undergo the following pharmacokinetic and dosimetry |
| assessments | assessments after receving the second [¹⁷⁷Lu]Lu-DOTA-TATE dose: |
| | Blood sampling for dosimetry on Week 5, Days 1, 2, 3 & 8 (Groups 1 + 2) |
| | and Week 4, Days 1, 2, 3 & 8 (Group 3) |
| | Whole body planar imaging on Week 5, Day 1, 2, 3 & 8 (Groups 1 + 2) |
| | and Week 4, Days 1, 2, 3 & 8 (Group 3) |
| | Urine collection for dosimetry on Week 5, Day 1 (Groups 1 + 2) and Week |
| | 4, Day 1 (Group 3) |
| | SPECT/CT imaging on Week 5, (Groups 1 + 2) and Week 4, (Group 3) in |
| | 24 hours after the end of [¹⁷⁷Lu]Lu-DOTA-TATE infusion |
| Key safety | Laboratory assessments including hematology, chemistry, urinalysis and |
| assessments | coagulation panel |
| | Physical and neurological examination |
| | Karnofsky Performance Status |
| | Vital signs |
| | Electrocardiogram (ECG) |
| | Pregnancy test |
| Other | Biomarker: Tumor and blood samples will be collected for biomarker analysis. |
| assessments | Tumor tissue: Newly obtained biopsy/resection or archival tissue block or |
| | slides at screening (mandatory for Groups 1 + 2, optional for Group 3). |
| | Participants with newly diagnosed glioblastoma will be assigned to Group |
| | 1 or 2 based on their MGMT promoter methylation status. Participants |
| | without existing local MGMT methylation test result can be tested locally, |
| | or submit 6-11 unstained formalin-fixed paraffin-embedded (FFPE) tumor |
| | tissue slides for centralized testing during screening. |
| | Blood: analysis will include circulating tumor DNA (ctDNA) and soluble |
| | protein biomarkers |
| Data analysis | The primary endpoint is the incidence of dose limiting toxicities (DLTs) during |
| | the DLT observation period, as defined for each group. |
| | Estimation of the recommended dose (RD) of the treatment will be based upon |
| | the estimation of the probability of DLT during the 49-52 days (depending on |
| | the start day of chemoradiation or radiotherapy) following the first administration |
| | (Day 1) of [¹⁷⁷Lu]Lu-DOTA-TATE in groups 1 and 2 or during the first 42 days |
| | in group 3. For the probability of estimating DLT, the Bayesian Optimal Interval |
| | Approach (BOIN) will be used for participants in the dose-determining set |
| | (DDS). |
| | The Dose-Determining Set (DDS) comprises all participants from the Full |
| | Analysis Set (FAS) who met the minimum exposure criterion and had sufficient |
| | safety evaluations, or experienced a dose limiting toxicity (DLT) during the DLT |
| | observation period. |
| | The minimum exposure criterion is defined as at least 90% of the planned dose |
| | of [¹⁷⁷Lu]Lu-DOTA-TATE for all groups, 75% of temozolomide dose for group 1 |
| | and 75% of radiotherapy for group 1 and Group 2. |
| | The primary analysis to support the declaration of the recommended dose will |
| | be performed once all participants have completed the DLT observation period. |
| | A further interim analysis will be performed when all participants across all |
| | groups have completed all planned [¹⁷⁷Lu]Lu-DOTA-TATE administrations or |
| | discontinued earlier. This analysis will assess the secondary endpoints of safety |
| | and preliminary activity of [¹⁷⁷Lu]Lu-DOTA-TATE in participants with newly |
| | diagnosed or recurrent glioblastoma. |
| | A final analysis to assess long-term safety and efficacy will be performed when |
| | all participants across all groups have completed the 52 weeks follow-up or |
| | discontinued earlier. |
| Primary Outcome | Group 1: Frequency of dose limiting toxicities (DLTs) [Time Frame: 49 to 52 |
| Measures: | days starting from first administration of [¹⁷⁷Lu]Lu-DOTA-TATE] |
| | A dose-limiting toxicity (DLT) is defined as an AE or abnormal laboratory value |
| | assessed as unrelated to disease, disease progression, inter-current illness, or |
| | concomitant medications that meets any of the criteria defining dose-limiting |
| | toxicities and with an onset during the DLT observation period specified for the |
| | group. |
| | Group 1: DLT observation period of 49 to 52 days starting from the first |
| | administration of [¹⁷⁷Lu]Lu-DOTA-TATE (Day 1) and ending with the completion |
| | of concomitant radiotherapy and TEMOZOLOMIDE. The 49 to 52 days |
| | observation period is depending on the start day of concomitant radiotherapy |
| | and temozolomide (i.e. 7-10 days post first [¹⁷⁷Lu]Lu-DOTA-TATE dose). If the |
| | concomitant radiotherapy and temozolomide is delayed for any reason, the DLT |
| | observation period will last until the completion of the concomitant treatment. If |
| | radiotherapy or temozolomide is delayed, the DLT observation period will last |
| | until the last administered dose, whichever occurs later. |
| | Group 2: Frequency of dose limiting toxicities (DLTs) [Time Frame: 49 to 52 |
| | days starting from first administration of [¹⁷⁷Lu]Lu-DOTA-TATE] |
| | A dose-limiting toxicity (DLT) is defined as an AE or abnormal laboratory value |
| | assessed as unrelated to disease, disease progression, inter-current illness, or |
| | concomitant medications that meets any of the criteria defining dose-limiting |
| | toxicities and with an onset during the DLT observation period specified for the |
| | group. |
| | Group 2: DLT observation period of 49 to 52 days starting from the first |
| | administration of [¹⁷⁷Lu]Lu-DOTA-TATE (Day 1) and ending with the completion |
| | of RT. The 49 to 52 days observation period is depending on the start day of |
| | radiotherapy (i.e. 7-10 days post first [¹⁷⁷Lu]Lu-DOTA-TATE dose). If |
| | radiotherapy is delayed for any reason, the DLT observation period will last until |
| | the completion of RT. |
| | Group 3: Frequency of dose limiting toxicities (DLTs) [Time Frame: 42 days |
| | starting from first administration of [¹⁷⁷Lu]Lu-DOTA-TATE] |
| | A dose-limiting toxicity (DLT) is defined as an AE or abnormal laboratory value |
| | assessed as unrelated to disease, disease progression, inter-current illness, or |
| | concomitant medications that meets any of the criteria defining dose-limiting |
| | toxicities and with an onset during the DLT observation period specified for the |
| | group. |
| | Group 3: DLT observation period of 42 days starting from the first |
| | administration of [¹⁷⁷Lu]Lu-DOTA-TATE (Day 1) i.e. accounting for 2 cycles of |
| | [¹⁷⁷Lu]Lu-DOTA-TATE. |
| Secondary | Incidence and severity of adverse events (AEs), serious AEs (SAEs) of |
| Outcome | [¹⁷⁷Lu]Lu-DOTA-TATE [Time Frame: From date of first study treatment until 8 |
| Measures | weeks after last treatment, assessed up approximately to 2 years (estimated |
| | final OS analysis)] |
| | The distribution of adverse events of [¹⁷⁷Lu]Lu-DOTA-TATE will be done via the |
| | analysis of frequencies for treatment emergent Adverse Event (TEAEs), |
| | Serious Adverse Event (TESAEs) and Deaths due to AEs, through the |
| | monitoring of relevant clinical and laboratory safety parameters. |
| | During the follow up period, all AEs and lab abnormalities will be collected within |
| | 8 weeks after the last dose of study treatment. For participants with study drug |
| | related AEs, they will be followed until resolution or until the End of Study, |
| | whichever occurs first. Apart from that, only survival information, SAEs, that are |
| | considered related to study drug by the Investigator, and AEs of secondary |
| | hematological malignancies will be collected until the end of follow up period |
| | every 8 weeks. |
| | Incidence and severity of Adverse Events (AEs) and serious Adverse |
| | Events (SAEs) within 48 hours after [⁶⁸Ga]Ga-DOTA-TATE infusion [Time |
| | Frame: up to 48 hours following the start of [⁶⁸Ga]Ga-DOTA-TATE |
| | administration] |
| | The distribution of adverse events of [⁶⁸Ga]Ga-DOTA-TATE will be done via the |
| | analysis of frequencies for treatment emergent Adverse Event (TEAEs), |
| | Serious Adverse Event (TESAEs) and Deaths due to AEs, through the |
| | monitoring of relevant clinical and laboratory safety parameters. |
| | Overall Objective Status as per modified Response Assessment in |
| | Neuro-Oncology (mRANO) criteria [Time Frame: From date of first treatment |
| | till 8 weeks safety follow-up, assessed up approximately to 2 years (estimated |
| | final OS analysis)] |
| | According to modified RANO, the objective overall status combines the |
| | radiographic response on target lesions, new disease, neurological status and |
| | use of steroids and the result is reported as confirmed or preliminary CR, |
| | confirmed or preliminary PR, stable disease, confirmed or preliminary disease |
| | progression. The best objective overall status achieved during the study will be |
| | summarized using frequency and percentage. The rate of confirmed complete |
| | or partial response will be summarized using point estimate and 2-sided exact |
| | binomial 95% confidence interval (Clopper-Pearson) and presented by dose |
| | level within each group. |
| | Progression-Free Survival (PFS) [Time Frame: Starting date of first |
| | treatment till approximately to 2 years (estimated final OS analysis)] |
| | Progression-Free Survival (PFS) is defined as the time from the date of first |
| | dose to the date of confirmed progression according to modified RANO or death |
| | due to any cause. If no PFS event is observed, PFS will be censored at the date |
| | of the last adequate tumor assessment prior to data cut-off date and start of |
| | new anti neoplastic therapy, whichever comes first. PFS will be analyzed in the |
| | FAS population and results will be presented for each dose level within each |
| | group. The PFS distribution will be estimated using the Kaplan-Meier method. |
| | Overall Survival (OS) [Time Frame: From date of first treatment till 8 |
| | weeks safety follow-up, assessed up approximately to 2 years (estimated final |
| | OS analysis)] |
| | Overall Survival (OS) is defined as the time from date of first dose to date of |
| | death due to any cause. If a participant is not known to have died, then OS will |
| | be censored at the latest date the participant was known to be alive (on or before |
| | the cut-off date). OS will be analyzed in the FAS population and results will be |
| | presented for each dose level within each group. The OS distribution will be |
| | estimated using the Kaplan-Meier method. |
| | Groups 1 and 2: Time activity curves (TACs) [Time Frame: Week 5 Day |
| | 1 (pre-dose, before the end of infusion, 2 hours post-dose, 6 hours post-dose), |
| | Week 5 Day 2 (24 hours post-dose), Week 5 Day 3 (48 hours post-dose), Week |
| | 5 Day 8 (168 hours post-dose)] |
| | Time activity curves (TACs), describing percentage (%) of the activity injected |
| | vs time in blood, organs and tumor lesions |
| | Groups 1 and 2: Absorbed radiation doses of [¹⁷⁷Lu]Lu-DOTA-TATE |
| | [Time Frame: Week 5 Day 1 (pre-dose, before the end of infusion, 2 hours post- |
| | dose, 6 hours post-dose), Week 5 Day 2 (24 hours post-dose), Week 5 Day 3 |
| | (48 hours post-dose), Week 5 Day 8 (168 hours post-dose)] |
| | Absorbed radiation doses of [¹⁷⁷Lu]Lu-DOTA-TATE in organs and tumor lesions |
| | will be summarized with descriptive statistics. |
| | Groups 1 and 2: Concentration of [¹⁷⁷Lu]Lu-DOTA-TATE [Time Frame: |
| | Week 5 Day 1 (pre-dose, before the end of infusion, 2 hours post-dose, 6 hours |
| | post-dose), Week 5 Day 2 (24 hours post-dose), Week 5 Day 3 (48 hours post- |
| | dose), Week 5 Day 8 (168 hours post-dose)] |
| | Blood samples for radioactivity measurement will be collected before the start |
| | of [¹⁷⁷Lu]Lu-DOTA-TATE infusion, at the end of [¹⁷⁷Lu]Lu-DOTA-TATE infusion, |
| | then at 2 h, 6 h, 24 h, 48 h and 168 h after the end of [¹⁷⁷Lu]Lu-DOTA-TATE |
| | infusion for participants undergoing dosimetry. Blood samples will be drawn in |
| | heparinized tubes. Radioactivity measurements on blood will be performed |
| | locally on site, using a gamma-counter. |
| | Groups 1 and 2: Observed maximum plasma concentration (Cmax) of |
| | [¹⁷⁷Lu]Lu-DOTA-TATE [Time Frame: Week 5 Day 1 (pre-dose, before the end |
| | of infusion, 2 hours post-dose, 6 hours post-dose), Week 5 Day 2 (24 hours |
| | post-dose), Week 5 Day 3 (48 hours post-dose), Week 5 Day 8 (168 hours post- |
| | dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Cmax will be listed and summarized using |
| | descriptive statistics. |
| | Groups 1 and 2: Time of maximum observed drug concentration |
| | occurrence (Tmax) of [¹⁷⁷Lu]Lu-DOTA-TATE [Time Frame: Week 5 Day 1 (pre- |
| | dose, before the end of infusion, 2 hours post-dose, 6 hours post-dose), Week |
| | 5 Day 2 (24 hours post-dose), Week 5 Day 3 (48 hours post-dose), Week 5 Day |
| | 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Tmax will be listed and summarized using |
| | descriptive statistics. |
| | Groups 1 and 2: Area under the serum concentration-time curve from |
| | time zero to the time of last quantifiable concentration (AUClast) of [¹⁷⁷Lu]Lu- |
| | DOTA-TATE [Time Frame: Week 5 Day 1 (pre-dose, before the end of infusion, |
| | 2 hours post-dose, 6 hours post-dose), Week 5 Day 2 (24 hours post-dose), |
| | Week 5 Day 3 (48 hours post-dose), Week 5 Day 8 (168 hours post-dose)] |
| | Venous whole blood samples will be collected and analysed for radioactivity. |
| | Radioactivity-based concentration units will be converted to mass-based |
| | concentration units using the specific activitiy of the dose at the time of |
| | manufacture (MBq/μg). Pharmacokinetics characterization will be performed on |
| | mass-based concentrations. AUClast will be listed and summarized using |
| | descriptive statistics. |
| | Groups 1 and 2: Area under the concentration-time curve from time zero |
| | (pre-dose) extrapolated to infinite time (AUCinf) of [¹⁷⁷Lu]Lu-DOTA-TATE [Time |
| | Frame: Week 5 Day 1 (pre-dose, before the end of infusion, 2 hours post-dose, |
| | 6 hours post-dose), Week 5 Day 2 (24 hours post-dose), Week 5 Day 3 (48 |
| | hours post-dose), Week 5 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. AUCinf will be listed and summarized using |
| | descriptive statistics. |
| | Groups 1 and 2: Total systemic clearance for intravenous administration |
| | (CL) of [¹⁷⁷Lu]Lu-DOTA-TATE [Time Frame: Week 5 Day 1 (pre-dose, before |
| | the end of infusion, 2 hours post-dose, 6 hours post-dose), Week 5 Day 2 (24 |
| | hours post-dose), Week 5 Day 3 (48 hours post-dose), Week 5 Day 8 (168 hours |
| | post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. CL will be listed and summarized using descriptive |
| | statistics. |
| | Groups 1 and 2: Volume of distribution during the terminal phase |
| | following intravenous elimination (Vz) of [¹⁷⁷Lu]Lu-DOTA-TATE [Time Frame: |
| | Week 5 Day 1 (pre-dose, before the end of infusion, 2 hours post-dose, 6 hours |
| | post-dose), Week 5 Day 2 (24 hours post-dose), Week 5 Day 3 (48 hours post- |
| | dose), Week 5 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Vz will be listed and summarized using descriptive |
| | statistics. |
| | Groups 1 and 2: Terminal elimination half-life (T\hat{ }½) of [¹⁷⁷Lu]Lu-DOTA- |
| | TATE [Time Frame: Week 5 Day 1 (pre-dose, before the end of infusion, 2 |
| | hours post-dose, 6 hours post-dose), Week 5 Day 2 (24 hours post-dose), Week |
| | 5 Day 3 (48 hours post-dose), Week 5 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. The half-life will be listed and summarized using |
| | descriptive statistics. |
| | Groups 1 and 2: Terminal-Phase Disposition Rate Constant (λz) of |
| | [¹⁷⁷Lu]Lu-DOTA-TATE [Time Frame: Week 5 Day 1 (pre-dose, before the end |
| | of infusion, 2 hours post-dose, 6 hours post-dose), Week 5 Day 2 (24 hours |
| | post-dose), Week 5 Day 3 (48 hours post-dose), Week 5 Day 8 (168 hours post- |
| | dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Terminal-Phase Disposition Rate Constant will be |
| | listed and summarized using descriptive statistics. |
| | Group 3: Time activity curves (TACs) [Time Frame: Week 4 Day 1 (pre- |
| | dose, before the end of infusion, 2 hours post-dose, 6 hours post-dose), Week |
| | 4 Day 2 (24 hours post-dose), Week 4 Day 3 (48 hours post-dose), Week 4 Day |
| | 8 (168 hours post-dose)] |
| | Time activity curves (TACs), describing percentage (%) of the activity injected |
| | vs time in blood, organs and tumor lesions |
| | Group 3: Absorbed radiation doses of [¹⁷⁷Lu]Lu-DOTA-TATE [Time |
| | Frame: Week 4 Day 1 (pre-dose, before the end of infusion, 2 hours post-dose, |
| | 6 hours post-dose), Week 4 Day 2 (24 hours post-dose), Week 4 Day 3 (48 |
| | hours post-dose), Week 4 Day 8 (168 hours post-dose)] |
| | Absorbed radiation doses of [¹⁷⁷Lu]Lu-DOTA-TATE in organs and tumor lesions |
| | will be summarized with descriptive statistics. |
| | Group 3: Concentration of [¹⁷⁷Lu]Lu-DOTA-TATE [Time Frame: Week 4 |
| | Day 1 (pre-dose, before the end of infusion, 2 hours post-dose, 6 hours post- |
| | dose), Week 4 Day 2 (24 hours post-dose), Week 4 Day 3 (48 hours post-dose), |
| | Week 4 Day 8 (168 hours post-dose)] |
| | Blood samples for radioactivity measurement will be collected before the start |
| | of [¹⁷⁷Lu]Lu-DOTA-TATE infusion, at the end of [¹⁷⁷Lu]Lu-DOTA-TATE infusion, |
| | then at 2 h, 6 h, 24 h, 48 h and 168 h after the end of [¹⁷⁷Lu]Lu-DOTA-TATE |
| | infusion for participants undergoing dosimetry. Blood samples will be drawn in |
| | heparinized tubes. Radioactivity measurements on blood will be performed |
| | locally on site, using a gamma-counter. |
| | Group 3: Observed maximum plasma concentration (Cmax) of [¹⁷⁷Lu]Lu- |
| | DOTA-TATE [Time Frame: Week 4 Day 1 (pre-dose, before the end of infusion, |
| | 2 hours post-dose, 6 hours post-dose), Week 4 Day 2 (24 hours post-dose), |
| | Week 4 Day 3 (48 hours post-dose), Week 4 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Cmax will be listed and summarized using |
| | descriptive statistics. |
| | Group 3: Time of maximum observed drug concentration occurrence |
| | (Tmax) of [¹⁷⁷Lu]Lu-DOTA-TATE [Time Frame: Week 4 Day 1 (pre-dose, before |
| | the end of infusion, 2 hours post-dose, 6 hours post-dose), Week 4 Day 2 (24 |
| | hours post-dose), Week 4 Day 3 (48 hours post-dose), Week 4 Day 8 (168 hours |
| | post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Tmax will be listed and summarized using |
| | descriptive statistics. |
| | Group 3: Area under the serum concentration-time curve from time zero |
| | to the time of last quantifiable concentration (AUClast) of [¹⁷⁷Lu]Lu-DOTA-TATE |
| | [Time Frame: Week 4 Day 1 (pre-dose, before the end of infusion, 2 hours post- |
| | dose, 6 hours post-dose), Week 4 Day 2 (24 hours post-dose), Week 4 Day 3 |
| | (48 hours post-dose), Week 4 Day 8 (168 hours post-dose)] |
| | Venous whole blood samples will be collected and analysed for radioactivity. |
| | Radioactivity-based concentration units will be converted to mass-based |
| | concentration units using the specific activity of the dose at the time of |
| | manufacture (MBq/μg). Pharmacokinetics characterization will be performed on |
| | mass-based concentrations. AUClast will be listed and summarized using |
| | descriptive statistics. |
| | Group 3: Area under the concentration-time curve from time zero (pre- |
| | dose) extrapolated to infinite time (AUCinf) of [¹⁷⁷Lu]Lu-DOTA-TATE [Time |
| | Frame: Week 4 Day 1 (pre-dose, before the end of infusion, 2 hours post-dose, |
| | 6 hours post-dose), Week 4 Day 2 (24 hours post-dose), Week 4 Day 3 (48 |
| | hours post-dose), Week 4 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. AUCinf will be listed and summarized using |
| | descriptive statistics. |
| | Group 3: Total systemic clearance for intravenous administration (CL) of |
| | [¹⁷⁷Lu]Lu-DOTA-TATE [Time Frame: Week 4 Day 1 (pre-dose, before the end |
| | of infusion, 2 hours post-dose, 6 hours post-dose), Week 4 Day 2 (24 hours |
| | post-dose), Week 4 Day 3 (48 hours post-dose), Week 4 Day 8 (168 hours post- |
| | dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. CL will be listed and summarized using descriptive |
| | statistics. |
| | Group 3: Volume of distribution during the terminal phase following |
| | intravenous elimination (Vz) of [¹⁷⁷Lu]Lu-DOTA-TATE [Time Frame: Week 4 |
| | Day 1 (pre-dose, before the end of infusion, 2 hours post-dose, 6 hours post- |
| | dose), Week 4 Day 2 (24 hours post-dose), Week 4 Day 3 (48 hours post-dose), |
| | Week 4 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Vz will be listed and summarized using descriptive |
| | statistics. |
| | Group 3: Terminal elimination half-life (T\hat{ }½) of [¹⁷⁷Lu]Lu-DOTA-TATE |
| | [Time Frame: Week 4 Day 1 (pre-dose, before the end of infusion, 2 hours post- |
| | dose, 6 hours post-dose), Week 4 Day 2 (24 hours post-dose), Week 4 Day 3 |
| | (48 hours post-dose), Week 4 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. The half-life will be listed and summarized using |
| | descriptive statistics. |
| | Group 3: Terminal-Phase Disposition Rate Constant (λz) of [¹⁷⁷Lu]Lu- |
| | DOTA-TATE [Time Frame: Week 4 Day 1 (pre-dose, before the end of infusion, |
| | 2 hours post-dose, 6 hours post-dose), Week 4 Day 2 (24 hours post-dose), |
| | Week 4 Day 3 (48 hours post-dose), Week 4 Day 8 (168 hours post-dose)] |
| | Venous whole blood samples will be collected and analysed for radioactivity. |
| | Radioactivity based concentration units will be converted to mass-based |
| | concentration units using the specific activity of the dose at the time of |
| | manufacture (MBq/μg). Pharmacokinetics characterization will be performed on |
| | mass-based concentrations. Terminal-Phase Disposition Rate Constant will be |
| | listed and summarized using descriptive statistics. |
| | Quantification of [¹⁷⁷Lu]Lu-DOTA-TATE excreted from the body in urine |
| | [Time Frame: From the start of [¹⁷⁷Lu]Lu-DOTA-TATE infusion until the first |
| | whole body planar imaging] |
| | All excreted urine from the start of [¹⁷⁷Lu]Lu-DOTA-TATE infusion until the first |
| | whole body planar imaging will be collected, its volume measured and the |
| | radioactivity concentration kilobecquerel per milliliter (kBq/mL) determined in |
| | order to calculate the total radioactivity excreted from the body from the start of |
| | [¹⁷⁷Lu]Lu-DOTA-TATE infusion to the time of the first scan. If no urine is |
| | excreted from the start of [¹⁷⁷Lu]Lu-DOTA-TATE infusion until the first whole |
| | body planar imaging, no urine dosimetry is necessary. |

The study for each participant consists of a Screening period, a Treatment period and a 12-month Follow-up period.

Eligible participants with newly diagnosed glioblastoma will be assigned to Group 1 or Group 2 depending on MGMT promoter methylation status:
Group 1: Newly diagnosed glioblastoma participants with methylated O-6-methylguanine-DNA methyltransferase (MGMT) promoter treated with [¹⁷⁷Lu]Lu-DOTA-TATE in combination with concomitant radiotherapy and temozolomide followed by [¹⁷⁷Lu]Lu-DOTA-TATE and temozolomide in maintenance.
Group 2: Newly diagnosed glioblastoma participants with unmethylated MGMT promoter treated with [¹⁷⁷Lu]Lu-DOTA-TATE in combination with radiotherapy followed by [¹⁷⁷Lu]Lu-DOTA-TATE alone.

Eligible participants with recurrent glioblastoma will be assigned to Group 3 and will receive [¹⁷⁷Lu]Lu-DOTA-TATE as single agent treatment.

| Arms | Assigned Interventions |
|---|---|
| Experimental: Group 1 - Newly diagnosed glioblastoma | Drug: [¹⁷⁷Lu]Lu-DOTA-TATE |
| (methylated MGMT) | Radiopharmaceutical solution for |
| Participants with newly diagnosed glioblastoma with | infusion (7.4 GBq per vial) |
| methylated MGMT, will receive [¹⁷⁷Lu]Lu-DOTA-TATE | Drug: [⁶⁸Ga]Ga-DOTA-TATE |
| every 4 weeks +/− 2 days, starting 7 to 10 days prior to | Kit for radiopharmaceutical |
| initiation of Radiotherapy (RT) and Temozolomide | preparation (40 mcg per kit) |
| (TEMOZOLOMIDE) | Other: Best supportive care |
| | Best supportive/best standard of |
| | care as defined by the local |
| | investigator |
| Experimental: Group 2 - Newly diagnosed glioblastoma | Drug: [¹⁷⁷Lu]Lu-DOTA-TATE |
| (unmethylated MGMT) | |
| Participants with newly diagnosed glioblastoma with | Radiopharmaceutical solution for |
| unmethylated MGMT, will receive [¹⁷⁷Lu]Lu-DOTA-TATE | infusion (7.4 GBq per vial) |
| every 4 weeks +/− 2 days for the first 3 doses (7 to 10 | Drug: [⁶⁸Ga]Ga-DOTA-TATE |
| days prior to initiation of Radiotherapy (RT) and at week | Kit for radiopharmaceutical |
| 4 and week 8 after Radiotherapy (RT) initiated) and | preparation (40 mcg per kit) |
| every 3 weeks +/− 2 days for the following doses | Other: Best supportive care |
| | Best supportive/best standard of |
| | care as defined by the local |
| | investigator |
| Experimental: Group 3 - Recurrent glioblastoma | Drug: [¹⁷⁷Lu]Lu-DOTA-TATE |
| Participants with recurrent glioblastoma will receive | Radiopharmaceutical solution for |
| [¹⁷⁷Lu]Lu-DOTA-TATE every 3 weeks +/− 2 days | infusion (7.4 GBq per vial) |
| | Drug: [⁶⁸Ga]Ga-DOTA-TATE |
| | Kit for radiopharmaceutical |
| | preparation (40 mcg per kit) |

The clinical study designs of the three groups are represented in the following:

1. A method of treating glioblastoma in a subject in need thereof comprising administering to said subject an efficient amount of a radiopharmaceutical compound in combination with a step of irradiating the subject with an efficient dose of ionizing radiations.

2. The method of claim 1, wherein said radiopharmaceutical compound is a compound of formula:
M-C—S—P wherein:
M is a radionuclide;
C is a chelating agent capable of chelating said radionuclide;
S is an optional spacer covalently linked between C and P;
P is a somatostatin receptor binding peptide covalently linked to C, either directly or indirectly via S.

3. The method of claim 1, wherein M is selected from ⁹⁰Y, ¹³¹I, ¹²¹Sn, ¹⁸⁶Re, ¹⁸⁸Re, ⁶⁴Cu, ⁶⁷Cu, ⁵⁹Fe, ⁸⁹Sr, ¹⁹⁸Au, ²⁰³Hg, ²¹²Pb, ¹⁶⁵Dy, ¹⁰³Ru, ¹⁴⁹Tb, ¹⁶¹Tb, ²¹³Bi, ¹⁶⁶Ho, ¹⁶⁵Er, ¹⁶⁹Er, ¹⁵³Sm, ¹⁷⁷Lu, ²¹³Bi, ²²³Ra, ²²⁵Ac, ²²⁷Ac, ²²⁷Th, ²¹¹At, ⁶⁷Cu, ¹⁸⁶Re, ¹⁸⁸Re, ¹⁶¹Tb, ¹⁷⁵Yb, ¹⁰⁵Rh, ¹⁶⁶Dy, ¹⁹⁹Au, ⁴⁴Sc, ¹⁴⁹Pm, ¹⁵¹Pm, ¹⁴²Pr, ¹⁴³Pr, ⁷⁶As, ¹¹¹Ag and ⁴⁷Sc, preferably is ¹⁷⁷Lu.

4. The method of claim 1, wherein C is selected from DOTA (tetraxetan), trizoxetan, DTPA, NTA, EDTA, DO3A, TETA, NOTA, NOTAGA, NODOGA, NODASA, NODAPA, and AAZTA (e.g. AAZTA5) chelating agent, preferably is DOTA, NOTA or DTPA chelating agent, and more preferably is DOTA chelating agent.

5. The method of claim 1, wherein P is selected from octreotide, octreotate, lanreotide, vapreotide, and pasireotide, preferably selected from octreotide and octreotate.

6. The method of claim 1, wherein the radiopharmaceutical compound is selected from DOTA-OC, DOTA-TOC (edotreotide), DOTA-NOC, DOTA-TATE (oxodotreotide), satoreotide tetraxetan, DOTA-LAN, and DOTA-VAP, preferably selected from DOTA-TOC and DOTA-TATE, more preferably is DOTA-TATE

7. The method of claim 1, wherein the radiopharmaceutical compound is [¹⁷⁷Lu]Lu-DOTA-TOC (¹⁷⁷Lu-edotreotide) or [¹⁷⁷Lu]Lu-DOTA-TATE (¹⁷⁷Lu-oxodotreotide), more preferably [¹⁷⁷Lu]Lu-DOTA-TATE (¹⁷⁷Lu-oxodotreotide).

8. The method of claim 1, said method further comprises administering a therapeutically efficient amount of an alkylating agent, preferably temozolomide.

9. The method of claim 1, wherein said alkylating agent, preferably temozolomide, is administered at an induction phase at a dose of between 50 to 100 mg/m²/day, preferably around 75 mg/m³/day each day for a period between 4 to 8 weeks, preferably 6 weeks.

10. The method of claim 1, wherein both irradiation and alkylating agent, preferably temozolomide, are initiated the same day.

11. The method of claim 1, wherein said alkylating agent, preferably temozolomide, is concomittantly administered with the irradiation without interruption.

12. The method of claim 1, wherein said alkylating agent, preferably temozolomide, is daily administered at a first dose during the concomitant administration with the radiotherapy, for example for a period of 6 weeks, and at a second dose during a maintenance phase, following the concomitant administration with the radiotherapy for example, for a period up to 24 weeks, wherein said second dose is at least twice the first dose.

13. The method of claim 1, wherein said alkylating agent, preferably temozolomide, is administered during the maintenance phase at a dose of between 50 to 400 mg/m²/day, preferably between 75 to 300 mg/m²/day, more preferably between 150 to 200 mg/m²/day each day for 5 consecutive days followed by 2 days of rest every 28 days for a period between 20 to 28 weeks, preferably 24 weeks.

14. The method of claim 1, wherein said subject has been selected from subject with positive methylated O-6-methylguanine-DNA methyltransferase promoter status.

15. The method of claim 1, wherein said radiopharmaceutical compound is administered at a dose ranging between 0.925 GBq (25 mCi) to 29.6 GBq (800 mCi), preferably between 1.48 GBq (40 mCi) to 18.5 GBq (500 mCi), preferably between 1.85 GBq (50 mCi) to 14.8 GBq (400 mCi), more preferably between 3.7 GBq (100 mCi) to 11.1 GBq (300 mCi), even more preferably of around 3.7 GBq (100 mCi), 5.55 GBq (150 mCi), 7.4 GBq (200 mCi) or 9.25 GBq (250 mCi).

16. The method of claim 1, wherein said radiopharmaceutical compound is administered 1 to 8 times per treatment at the induction phase, preferably 2 to 7 times per treatment, more preferably 4 to 6 times per treatment.

17. The method of claim 1, wherein said radiopharmaceutical compound comprises a treatment interval of 2 weeks, or 3 weeks, or 4 weeks, or 5 weeks or even 6 weeks, preferably 3 or 4 weeks, more preferably every 3 weeks.

18. The method of claim 1, wherein a first dose of said radiopharmaceutical compound is administered 1 to 20 days, preferably 3 to 15 days, more preferably 7 to 10 days prior to initiation of irradiation.

19. The method of claim 1, wherein said irradiation induction is conducted at a dose between 1 Gy to 4 Gy/day, preferably around 2 Gy/day during a period between 3 to 7 days, preferably around 5 days per week during a period between 4 to 8 weeks, preferably 6 weeks.

20. The method of claim 1, wherein said irradiation is conducted for 5 consecutive days followed by 2 days of rest for 6 consecutive weeks.

21. The method of claim 1, wherein said irradiation is whole-brain irradiation.

22. The method of claim 1, wherein said subject has been selected for the treatment by SPECT/CT or PET/CT or SPECT/MRI, PET/MRI imaging with the same radiopharmaceutical compound as defined for the treatment but with a radiometal suitable for imaging instead of ¹⁷⁷Lu, preferably ⁶⁸Ga, ⁶⁷Ga or ⁶⁴Cu, more preferably ⁶⁸Ga, by evaluating said radiopharmaceutical compound suitable for imaging uptake in said subject.

23. The method of claim 1, wherein said subject is newly diagnosed with glioblastoma or suffers from recurrent glioblastoma.

24. The method of claim 23, wherein said subject is newly diagnosed with glioblastoma and has a positive methylated O-6-methylguanine-DNA methyltransferase promoter status, wherein said radiopharmaceutical compound is administered to said subject in combination with radiotherapy and an alkylating agent, preferably temozolomide, wherein a first dose of said radiopharmaceutical compound is administered preferably 7 to 10 days prior to initiation of radiotherapy.

25. The method of claim 23, wherein said subject is newly diagnosed with glioblastoma and has a negative methylated O-6-methylguanine-DNA methyltransferase promoter status, wherein said radiopharmaceutical compound is administered to said subject in combination with radiotherapy and wherein not in combination with other chemotherapeutic agent, such as temozolomide; and wherein the treatment interval between two administrations of said radiopharmaceutical compound is for the first two intervals 4 weeks, and for the third and any following intervals, 3 weeks; wherein a first dose of said radiopharmaceutical compound is administered preferably 7 to 10 days prior to initiation of radiotherapy.
